(19) United States
(12) Patent Application Publication
(10) Pub. No.: US 2026/0098335 A1
(43) Pub. Date: Apr. 9, 2026

(54) PRECURSOR FOR FORMING THIN FILM COMPRISING AMIDINATE LIGAND

(71) Applicant: SK TRI CHEM CO., LTD., Sejong (KR)

(72) Inventors: Han Byul KIM, Sejong (KR); Han Sol OH, Sejong (KR); Yong Joo PARK, Sejong (KR)

(21) Appl. No.: 19/415,731

(22) Filed: Dec. 10, 2025

Related U.S. Application Data
(63) Continuation of application No. PCT/KR2025/002787, filed on Feb. 28, 2025.

(30) Foreign Application Priority Data
Feb. 28, 2024 (KR) 10-2024-0028858
Feb. 27, 2025 (KR) 10-2025-0025961

(51) Int. Cl. C23C 16/18; C07F 5/00

(52) U.S. Cl. CPC C23C 16/18; C07F 5/00

(57) ABSTRACT

Proposed is a precursor for forming a thin film including a compound represented by Chemical Formula 1, wherein the precursor for forming a thin film is a liquid at room temperature, and contains an amidinate ligand capable of exhibiting chemical properties of the precursor, such as high structural stability, low viscosity, high volatility, high heat resistance, and room-temperature liquid form.

### TECHNICAL FIELD

The present disclosure relates to a precursor for forming a thin film including an amidinate ligand. More specifically, the present disclosure relates to a precursor for forming a thin film including an amidinate ligand, wherein the precursor contains a novel amidinate ligand, thereby forming a precursor that exhibits low viscosity, high heat resistance, and high volatility, and is a liquid at room temperature to enable the formation of a high-quality thin film.

### BACKGROUND ART

With improved integration density achieved through the miniaturization of line widths of semiconductor devices, the space allowed for implementing capacitor structures has become limited. Accordingly, there have been limitations in using existing methods in manufacturing methods for semiconductor devices designed to implement capacitor structures. In particular, with the application of high-dielectric thin films, there have been issues where the leakage current caused by the band gap of such high-dielectric thin films is significantly increased, resulting in degradation. One of the solutions for addressing these issues requires a technology for forming high-quality thin films, which necessitates the optimization of precursors used in thin film formation for that purpose.

A precursor for forming a thin film is composed of a central metal atom and a ligand. Because chemical properties such as viscosity, heat resistance, and volatility vary depending on the structure of this ligand, precursors to which various types of ligands are bound have been developed in consideration of such factors.

For example, known technologies, including Korean Patent No. 10-1660052, Korean Patent Application Publication No. 10-2019-0109142, and Korean Patent Application Publication No. 10-2021-0084297, in the art have proposed chemical structures serving as yttrium- or lanthanide metal-containing precursors, to which cyclopentadienyl and amidinate are bound as ligands. It has been reported that the precursors to which these ligands are bound can overcome the disadvantages of existing yttrium or lanthanide metal precursors having low vapor pressure and high viscosity, and thus are suitable for use in thin-film formation processes.

However, such conventional precursors may still cause various problems in semiconductor thin-film formation processes due to having high viscosity. Therefore, it is necessary to develop a precursor that exhibits chemical properties (such as being a liquid, highly volatile, and highly heat-resistant) required for precursors in semiconductor thin-film formation processes while improving viscosity. In particular, referring to the results of known technologies, a chemical structure containing an amidinate as a ligand is expected to obtain the chemical properties required for a precursor used in thin-film formation processes.

### DISCLOSURE

### Technical Problem

The present disclosure, which has been devised in view of technologies in the art as described above, aims to provide a precursor for forming a thin film including a novel amidinate ligand.

The present disclosure also aims to provide a precursor for forming a thin film including the above ligand, thereby exhibiting chemical properties such as low viscosity, high heat resistance, and high volatility.

### Technical Solution

A precursor for forming a thin film of the present disclosure, which is provided to achieve the objectives as described above, is characterized by including a compound represented by Chemical Formula 1 below, wherein the precursor is a liquid at room temperature.

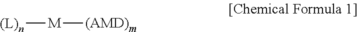

In Chemical Formula 1, AMD refers to an amidinate ligand, M is a central metal atom and is any one of the following: a Group 2 to 6 element, a Group 13 element, a Group 15 element, a transition metal, and a rare-earth element, L is a ligand that is the same as or different from AMD and, when being different from AMD, is any one of the following: a substituted or unsubstituted cyclopentadienyl group, amine, alcohol, alkyl, aryl, amino amine, alkoxy amine, amino alcohol, alkoxy alcohol, imido, diamine, diol, formidinate, guanidinate, β-diketonate, ketoiminate, amide, or halide, n is an integer in a range of 0 to 5, and m is an integer in a range of 1 to 6.

In this case, AMD may be a ligand represented by Chemical Formula 2 below.

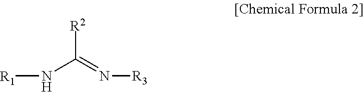

In Chemical Formula 2, R₁ and R₃ are each independently a straight-chain, branched-chain, or cyclic alkyl or alkenyl group having 1 to 5 carbon atoms, and R₂ is a hydrogen atom, or a straight-chain, branched-chain, or cyclic alkyl or alkenyl group having 1 to 5 carbon atoms.

Additionally, in Chemical Formula 2, R₂ may be a straight-chain, branched-chain, or cyclic alkyl or alkenyl group having 2 to 5 carbon atoms.

Additionally, in Chemical Formula 2, R₁ and R₃ may each independently be a straight-chain, branched-chain, or cyclic alkyl or alkenyl group having 2 to 5 carbon atoms, and R₂ may be a straight-chain, branched-chain, or cyclic alkyl or alkenyl group having 2 to 5 carbon atoms.

Additionally, in Chemical Formula 2, R₁ and R₃ may be methyl groups.

Additionally, in Chemical Formula 2, R₂ may be an isopropyl group.

Additionally, in Chemical Formula 2, R₁ and R₃ may each independently be a straight-chain alkyl or alkenyl group having 1 to 5 carbon atoms.

Additionally, in Chemical Formula 2, R₁ and R₃ may each independently be a straight-chain alkyl or alkenyl group having 1 to 5 carbon atoms, and R₂ may be a straight-chain alkyl or alkenyl group having 1 to 5 carbon atoms.

Additionally, in Chemical Formula 2, R₁ and R₃ may both be the same, and may be straight-chain, branched-chain, or cyclic alkyl or alkenyl groups having 1 to 5 carbon atoms.

Additionally, in Chemical Formula 2, R₁ to R₃ may all be the same, and are straight-chain, branched-chain, or cyclic alkyl or alkenyl groups having 1 to 4 carbon atoms.

Additionally, the precursor for forming a thin film may be a precursor having a low viscosity of 60 cP or less (at a temperature of 25° C.).

### Advantageous Effects

A precursor for forming a thin film, according to the present disclosure, contains an amidinate ligand, thereby enabling chemical properties of the precursor compound, such as high structural stability, low viscosity, high volatility, high heat resistance, and room-temperature liquid form, to be exhibited.

Therefore, the precursor for forming a thin film, including the above amidinate ligand, exhibits properties suitable for use in a thin-film formation process and thus enables the formation of a high-quality thin film, without issues of thermal decomposition during the process or residues in the pipe resulting from high viscosity. Additionally, a semiconductor device including a thin film manufactured by the thin-film formation method can be provided.

### DESCRIPTION OF DRAWINGS

FIG. 1 shows the proton nuclear magnetic resonance (¹H-NMR) result of a diethyl-ethylamidinate ligand.

FIG. 2 shows the thermogravimetric analysis (TGA) result of a diethyl-ethylamidinate ligand.

FIG. 3 shows the ¹H-NMR result of a diethyl-n-propylamidinate ligand.

FIG. 4 shows the TGA result of a diethyl-n-propylamidinate ligand.

FIG. 5 shows the ¹H-NMR result of a di-n-propyl-ethylamidinate ligand.

FIG. 6 shows the TGA result of a di-n-propyl-ethylamidinate ligand.

FIG. 7 shows the ¹H-NMR result of bis(ethylcyclopentadienyl)(diethyl-ethylamidinato)yttrium.

FIG. 8 shows the TGA result of bis(ethylcyclopentadienyl)(diethyl-ethylamidinato)yttrium.

FIG. 9 shows the differential scanning calorimetry (DSC) result of bis(ethylcyclopentadienyl)(diethyl-ethylamidinato)yttrium.

FIG. 10 shows the ¹H-NMR result of bis(ethylcyclopentadienyl)(diethyl-n-propylamidinato)yttrium.

FIG. 11 shows the TGA result of bis(ethylcyclopentadienyl)(diethyl-n-propylamidinato)yttrium.

FIG. 12 shows the ¹H-NMR result of bis(isopropylcyclopentadienyl)(diethyl-ethylamidinato)yttrium.

FIG. 13 shows the ¹H-NMR result of bis(methylcyclopentadienyl)(diethyl-n-propylamidinato)yttrium.

FIG. 14 shows the TGA result of bis(methylcyclopentadienyl)(diethyl-n-propylamidinato)yttrium.

FIG. 15 shows the ¹H-NMR result of bis(methylcyclopentadienyl)(di-n-propyl-ethylamidinato)yttrium.

FIG. 16 shows the TGA result of bis(methylcyclopentadienyl)(di-n-propyl-ethylamidinato)yttrium.

FIG. 17 shows the ¹H-NMR result of bis(ethylcyclopentadienyl)(dimethyl-propylamidinato)yttrium.

FIG. 18 shows the TGA result of bis(ethylcyclopentadienyl)(dimethyl-propylamidinato)yttrium.

FIG. 19 shows the DSC result of bis(ethylcyclopentadienyl)(dimethyl-propylamidinato)yttrium.

FIG. 20 shows the ¹H-NMR result of bis(ethylcyclopentadienyl)(diethyl-n-propylamidinato)scandium.

FIG. 21 shows the TGA result of bis(ethylcyclopentadienyl)(diethyl-n-propylamidinato)scandium.

FIG. 22 shows the TGA result of bis(isopropylcyclopentadienyl)(diethyl-n-propylamidinato)cesium.

FIG. 23 shows the DSC result of bis(isopropylcyclopentadienyl)(diethyl-n-propylamidinato)cesium.

FIG. 24 shows the TGA result of bis(ethylcyclopentadienyl)(diethyl-ethylamidinato)gadolinium.

FIG. 25 shows the DSC result of bis(ethylcyclopentadienyl)(diethyl-ethylamidinato)gadolinium.

FIG. 26 shows the TGA result of bis(ethylcyclopentadienyl)(diethyl-ethylamidinato)dysprosium.

FIG. 27 shows the DSC result of bis(ethylcyclopentadienyl)(diethyl-ethylamidinato)dysprosium.

FIG. 28 shows the ¹H-NMR result of bis(ethylcyclopentadienyl)(diethyl-ethylamidinato)lutetium.

FIG. 29 shows the TGA result of bis(ethylcyclopentadienyl)(diethyl-ethylamidinato)lutetium.

FIG. 30 shows the DSC result of bis(ethylcyclopentadienyl)(diethyl-ethylamidinato)lutetium.

FIG. 31 shows the ¹H-NMR result of tris(diethyl-n-propylamidinato)yttrium.

FIG. 32 shows the TGA result of tris(diethyl-n-propylamidinato)yttrium.

FIG. 33 shows the TGA result of tris-(diethyl-n-propylamidinato)terbium.

FIG. 34 shows the DSC result of tris-(diethyl-n-propylamidinato)terbium.

FIG. 35 shows the TGA result of tris-(diethyl-n-propylamidinato)dysprosium.

FIG. 36 shows the DSC result of tris-(diethyl-n-propylamidinato)dysprosium.

FIG. 37 shows the TGA result of tris-(diethyl-n-propylamidinato)erbium.

FIG. 38 shows the DSC result of tris-(diethyl-n-propylamidinato)erbium.

FIG. 39 shows the TGA result of tris-(diethyl-n-propylamidinato)ytterbium.

FIG. 40 shows the ¹H-NMR result of tris-(diethyl-n-propylamidinato)lutetium.

FIG. 41 shows the TGA result of tris-(diethyl-n-propylamidinato)lutetium.

FIG. 42 shows the DSC result of tris-(diethyl-n-propylamidinato)lutetium.

FIG. 43 shows the ¹H-NMR result of (CH₂CH₂CH₂N—C(CH₂CH₃)=N—CH₂CH₂CH₃)₂Hf(DMA)₂.

FIG. 44 shows the TGA and DSC results of (CH₂CH₂CH₂N—C(CH₂CH₃)=N—CH₂CH₂CH₃)₂Hf(DMA)₂ in (a) and (b), respectively.

FIG. 45 shows the ¹H-NMR result of (CH₂CH₂N—C(CH₂CH₂CH₃)=N—CH₂CH₃)₂If(DMA)₂.

FIG. 46 shows the TGA and DSC results of (CH₂CH₂N—C(CH₂CH₂CH₃)=N—CH₂CH₃)₂If(DMA)₂ in (a) and (b), respectively.

FIG. 47 shows the ¹H-NMR result of (CH₂CH₂N—C(CH₂CH₂CH₃)=N—CH₂CH₃)₂Hf(EMA)₂.

FIG. 48 shows the TGA and DSC results of (CH₂CH₂N—C(CH₂CH₂CH₃)=N—CH₂CH₃)₂Hf(EMA)₂ in (a) and (b), respectively.

FIG. 49 shows the ¹H-NMR result of (CH₂CH₂N—C(CH₂CH₂CH₃)=N—CH₂CH₃)₂Zr(DMA)₂.

FIG. 50 shows the TGA and DSC results of (CH₂CH₂N—C(CH₂CH₂CH₃)=N—CH₂CH₃)₂Zr(DMA)₂ in (a) and (b), respectively.

FIG. 51 shows the ¹H NMR result of (2-ethyl-N,N-diethylamidinato)bis(dimethylamino)borane.

FIG. 52 shows the ¹¹B NMR result of (2-ethyl-N,N-diethylamidinato)bis(dimethylamino)borane.

FIG. 53 shows the ¹H NMR result of (2-methyl-N,N-diisopropylamidinato)bis(dimethylamino)borane.

FIG. 54 shows the ¹B NMR result of (2-methyl-N,N-diisopropylamidinato)bis(dimethylamino)borane.

FIG. 55 shows the ¹H NMR result of (2-ethyl-N,N-diethylamidinato)(N,N-dimethylethylenediamino)borane.

FIG. 56 shows the ¹¹B NMR result of (2-ethyl-N,N-diethylamidinato)(N,N-dimethylethylenediamino)borane.

FIG. 57 shows the ¹H NMR result of (N-ethyl-2-isobutyl-N-propylamidinato)bis(dimethylamino)borane.

FIG. 58 shows the ¹¹B NMR result of (N-ethyl-2-isobutyl-N-propylamidinato)bis(dimethylamino)borane.

### MODE FOR INVENTION

Hereinafter, the present disclosure will be described in more detail. All terms or words used in this specification and the appended claims should not be construed as being limited to general and dictionary meanings, but will be interpreted based on the meanings and concepts corresponding to the technical ideas of the present disclosure, following the principle that any inventor is allowed to define the concepts of terms as appropriate to describe the disclosure thereof in the best mode.

A precursor for forming a thin film, according to the present disclosure, is characterized by including a compound represented by Chemical Formula 1 below, wherein the precursor is a liquid at room temperature.

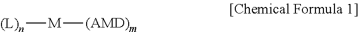

In Chemical Formula 1, AMD refers to an amidinate ligand, M is a central metal atom and is any one of the following: a Group 2 to 6 element, a Group 13 element, a Group 15 element, a transition metal, and a rare-earth element, L is a ligand that is the same as or different from AMD and, when being different from AMD, is any one of the following: a substituted or unsubstituted cyclopentadienyl group, amine, alcohol, alkyl, aryl, amino amine, alkoxy amine, amino alcohol, alkoxy alcohol, imido, diamine, diol, formidinate, guanidinate, β-diketonate, ketoiminate, amide, or halide, n is an integer in the range of 0 to 5, and m is an integer in the range of 1 to 6.

Additionally, as for AMD, a ligand compound represented by Chemical Formula 2 below may be applied.

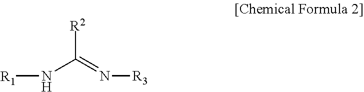

In Chemical Formula 2, R₁ and R₃ are each independently a straight-chain, branched-chain, or cyclic alkyl or alkenyl group having 1 to 5 carbon atoms, and R₂ is a hydrogen atom, or a straight-chain, branched-chain, or cyclic alkyl or alkenyl group having 1 to 5 carbon atoms.

The amidinate ligand is used as a ligand of the precursor compound for forming a thin film of the present disclosure. In particular, R₁ and R₃ in the amidinate ligand may be the same or different, and the ligand may be configured in various forms depending on the desired effect of the precursor containing the ligand.

Additionally, while R₂ in Chemical Formula 2 may be a hydrogen atom, or a straight-chain, branched-chain, or cyclic alkyl or alkenyl group having 1 to 5 carbon atoms, non-limiting examples of R₂ may include an n-alkyl group, such as an ethyl group, a propyl group, or a butyl group.

The precursor containing the amidinate ligand represented by Chemical Formula 2 has low viscosity, high thermal stability, and high volatility, and may be in liquid form at room temperature. Therefore, it has been found that the chemical properties of a desired precursor can be obtained by synthesizing the precursor containing the ligand. In other words, the precursor exhibits overall enhanced physical properties compared to various precursor compounds containing amidinate ligands in the art. As a result, not only can the effectiveness of the precursor be improved, but also enhanced effects can be obtained in a thin-film formation process using the precursor.

The amidinate ligand represented by Chemical Formula 2 can form structures in which various functional groups are combined.

In one embodiment, R₂ may be a straight-chain, branched-chain, or cyclic alkyl or alkenyl group having 2 to 5 carbon atoms. Compared to being H or having 1 carbon atom, R₂ provided with 2 or more carbon atoms can reduce intermolecular interference by increasing structural asymmetry while minimizing intramolecular structural steric hindrance. Therefore, the precursor containing the ligand can not only be easily provided in a liquid form, but also can exhibit properties such as low viscosity.

In the meantime, R₁ and R₃ may each independently be a straight-chain, branched-chain, or cyclic alkyl or alkenyl group having 2 to 5 carbon atoms, and R₂ may be a straight-chain, branched-chain, or cyclic alkyl or alkenyl group having 2 to 5 carbon atoms.

In a further embodiment, R₁ and R₃ may be methyl groups, and R₂ may be an isopropyl group.

Additionally, R₁ and R₃ may each independently be a straight-chain alkyl or alkenyl group having 1 to 5 carbon atoms.

The molecular weight of straight-chain alkyl or alkenyl groups may be reduced by making ligand structures the smallest compared to branched-chain or cyclic alkyl or alkenyl groups, thereby obtaining the effects of improving volatility and vapor pressure. Therefore, in the ligand according to one embodiment of the present disclosure, R₁ and R₃ may each independently be a straight-chain alkyl or alkenyl group. In this case, the precursor for forming a thin film, including the above ligand, exhibits increased vapor pressure, which may provide effects such as improved processability during the thin-film formation process using the precursor.

In addition to the effect of increasing vapor pressure, the ligand in which R₁ and R₃ are each independently a straight-chain alkyl group exhibits high structural flexibility, so the effect of increasing the flexibility of the precursor employing the above ligand can be provided. Such an effect minimizes interference between the precursors, thereby providing the effects of liquefying the precursor and imparting properties such as low viscosity.

Thus, in the amidinate ligand according to one embodiment, R₁ and R₃ in Chemical Formula 2 may be straight-chain alkyl or alkenyl groups, in which case the precursor containing the above ligand may exhibit increased vapor pressure. Furthermore, the increased flexibility can not only facilitate formation in a liquid state, but also enable the precursor to exhibit properties such as low viscosity.

In the meantime, R₁ and R₃ may each independently be a straight-chain alkyl or alkenyl group having 1 to 5 carbon atoms, and R₂ may be a straight-chain alkyl or alkenyl group having 1 to 5 carbon atoms.

Additionally, R₁ and R₃ may both be the same, and may be straight-chain, branched-chain, or cyclic alkyl or alkenyl groups having 1 to 5 carbon atoms.

Additionally, R₁ to R₃ may all be the same, and may be straight-chain, branched-chain, or cyclic alkyl or alkenyl groups having 1 to 4 carbon atoms.

Exemplary structures of such an amidinate ligand may include one or more selected from the following chemical structures.

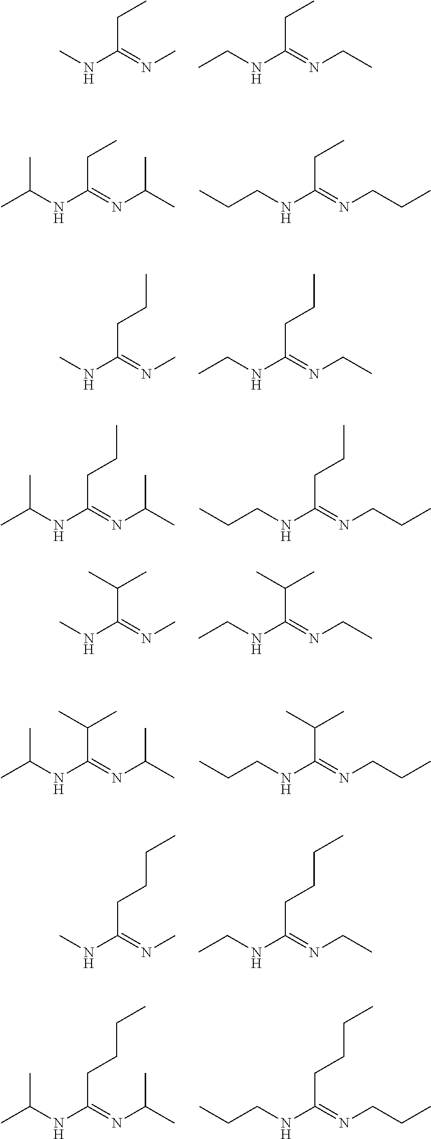

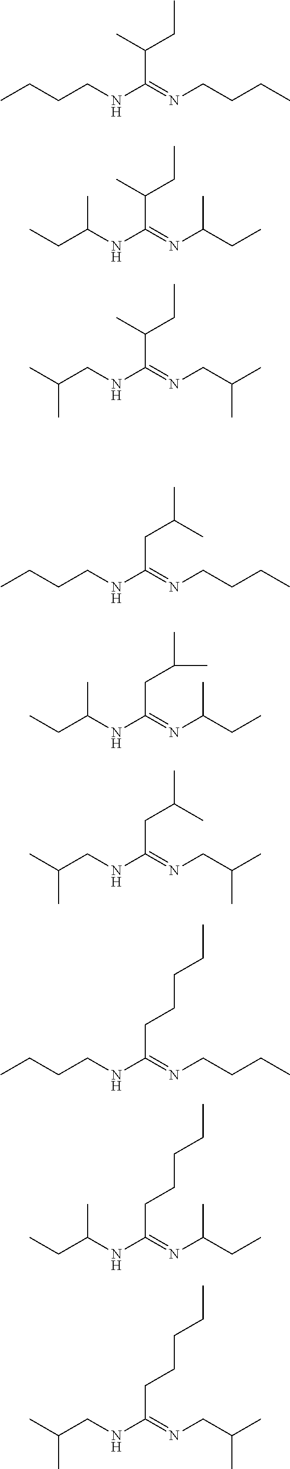

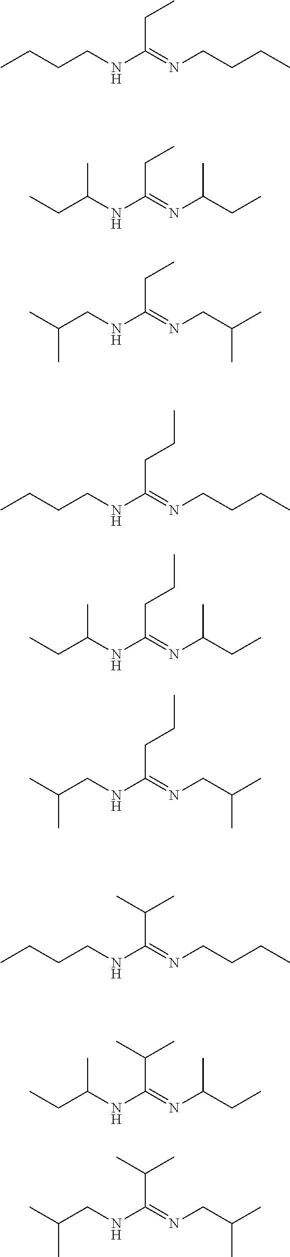

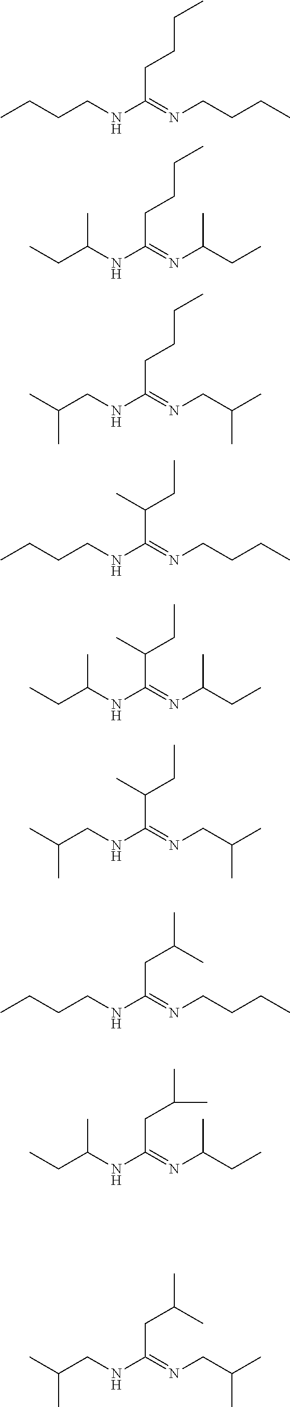

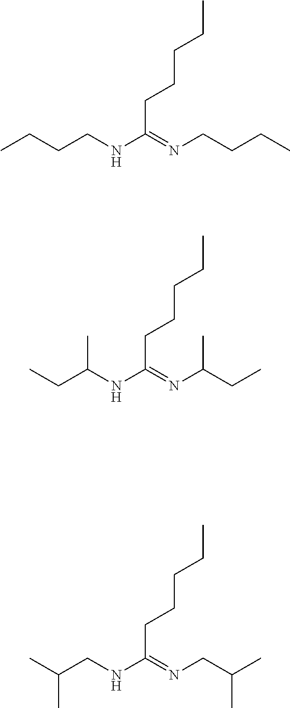

In the meantime, the precursor for forming a thin film including the above amidinate ligand may have a central metal atom being any one of the following: a Group 2 to 6 element, a Group 13 element, a Group 15 element, a transition metal, and a rare-earth element.

Non-limiting examples of the central metal atom may include yttrium (Y), scandium (Sc), or any one of the lanthanide elements.

Additionally, non-limiting examples of the precursor for forming a thin film, which may be represented by Chemical Formula 1, include compounds represented by various chemical structures as follows.

For example, when L is a compound containing a substituted or unsubstituted cyclopentadienyl group, the following compounds may be exemplified.

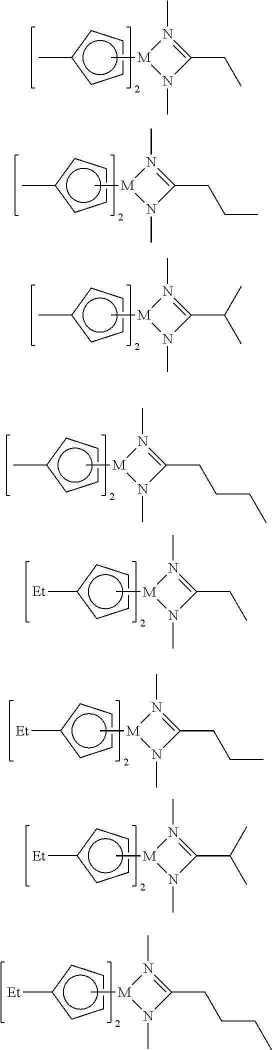

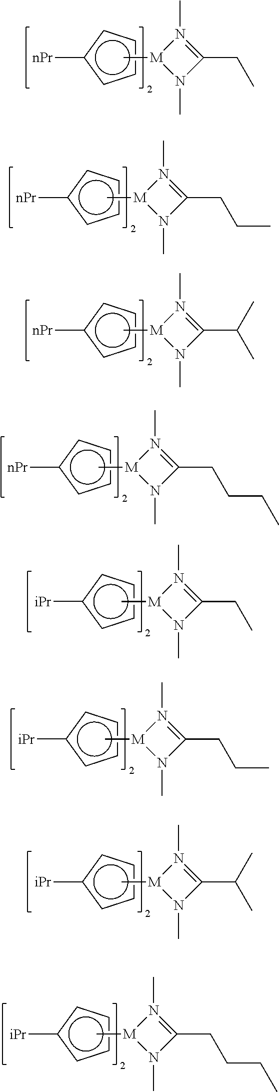

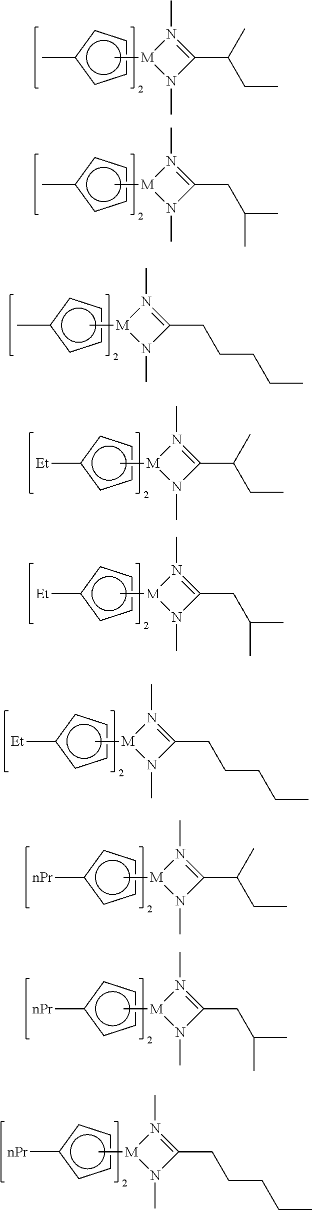

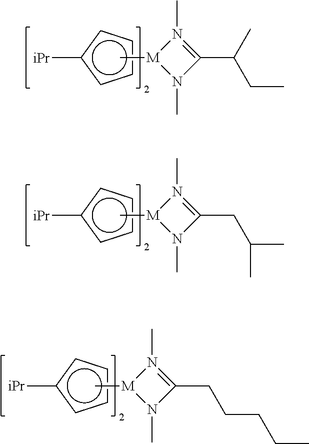

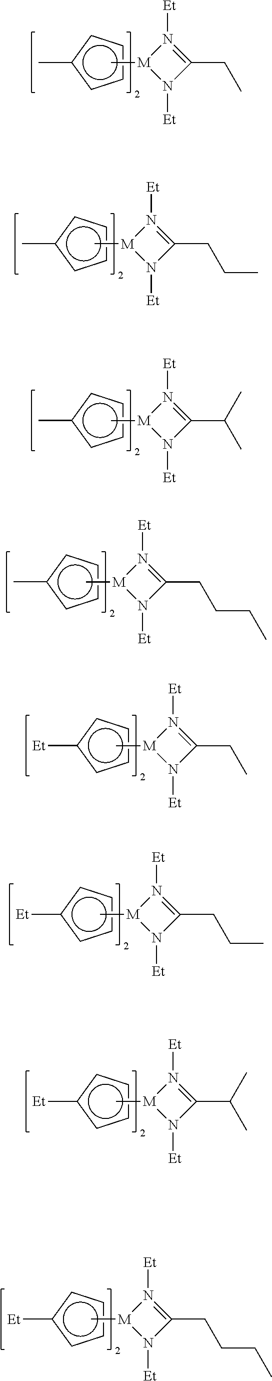

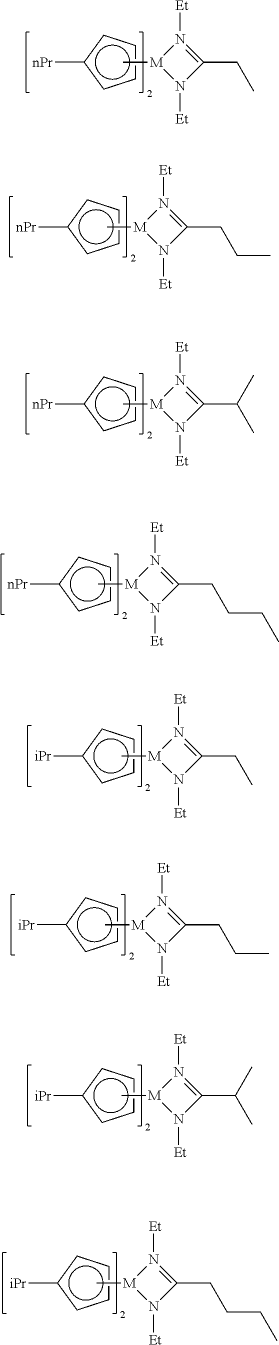

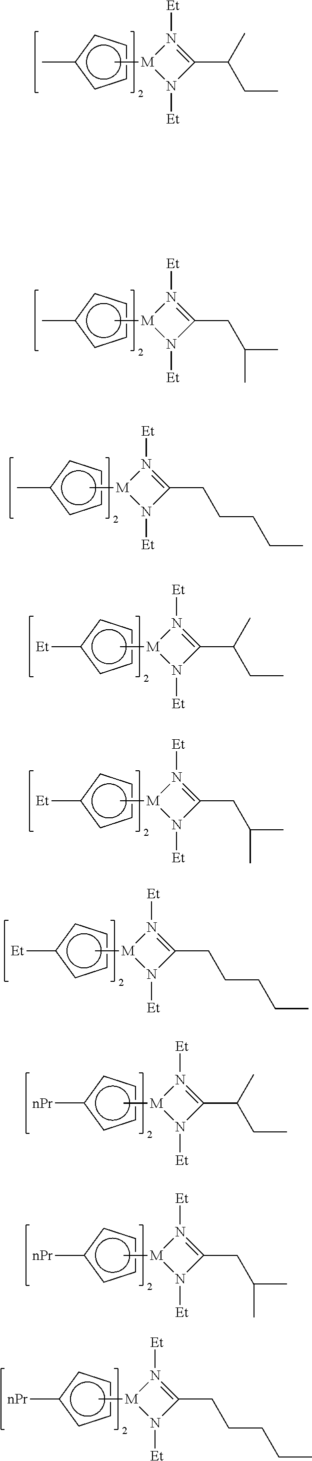

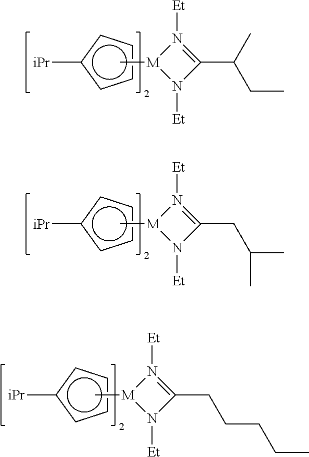

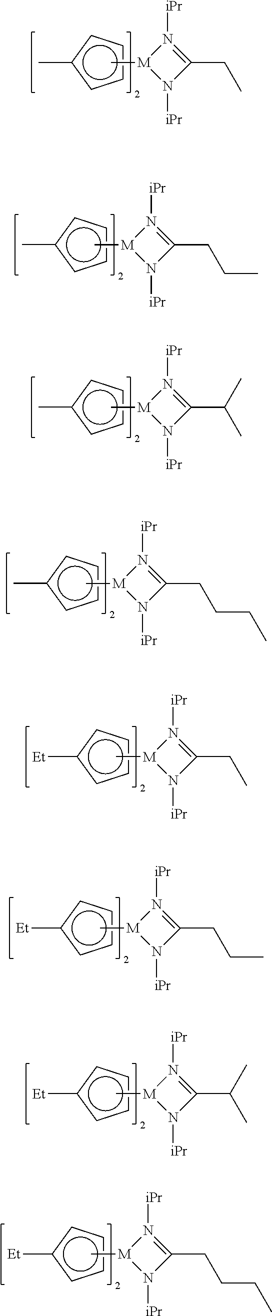

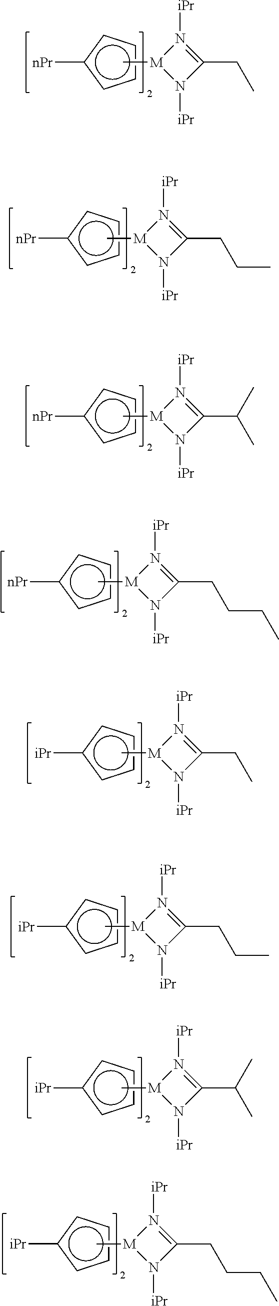

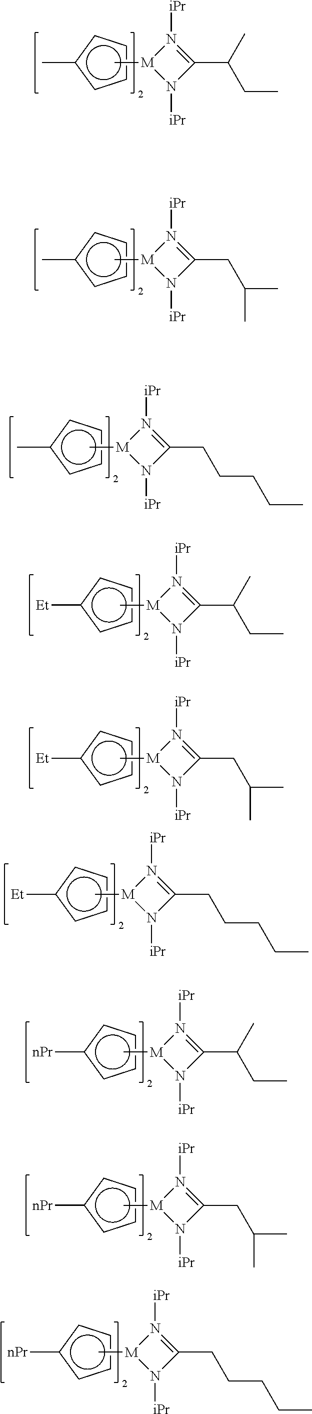

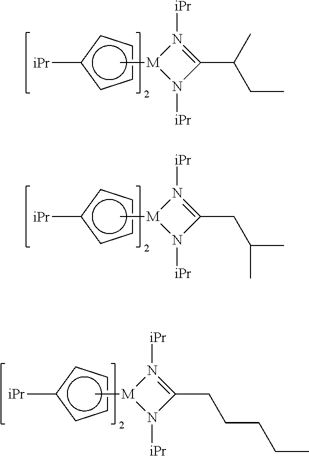

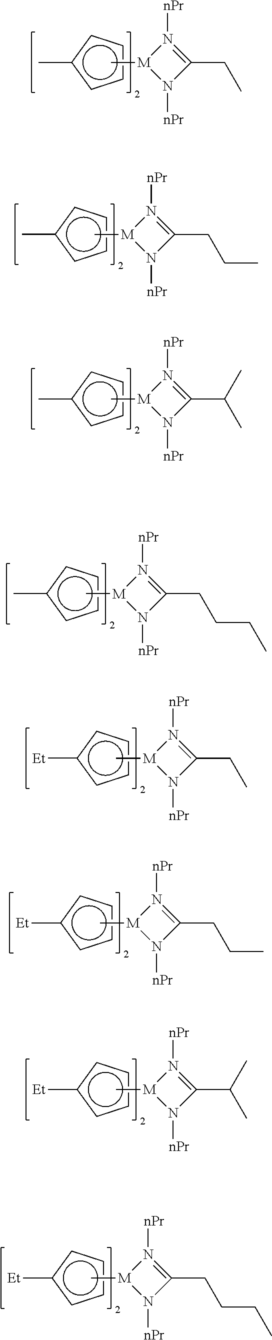

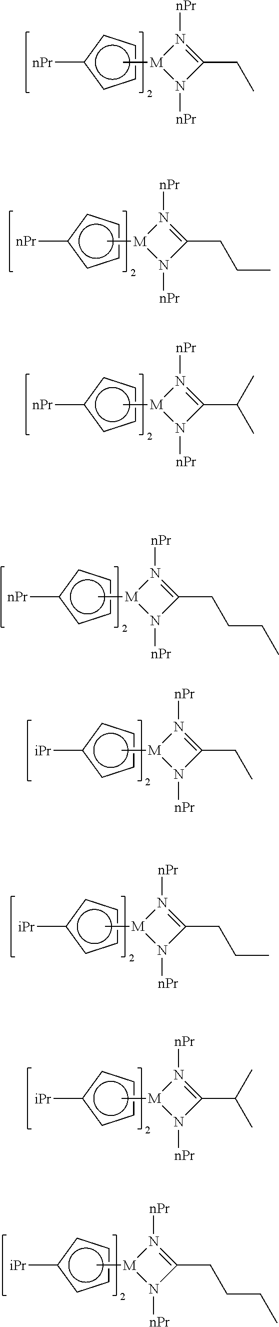

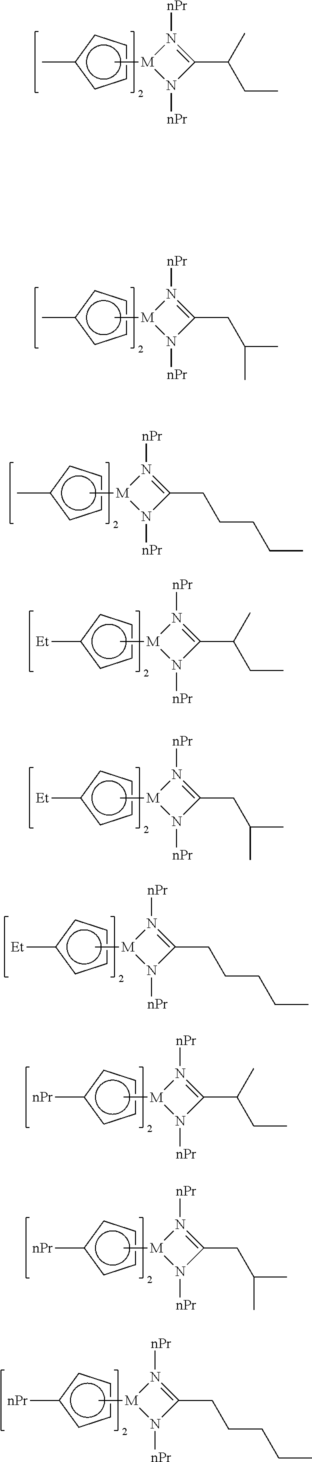

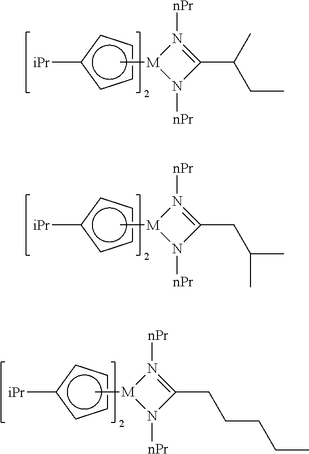

Additionally, when Lis an amine group, the following compounds may be exemplified.

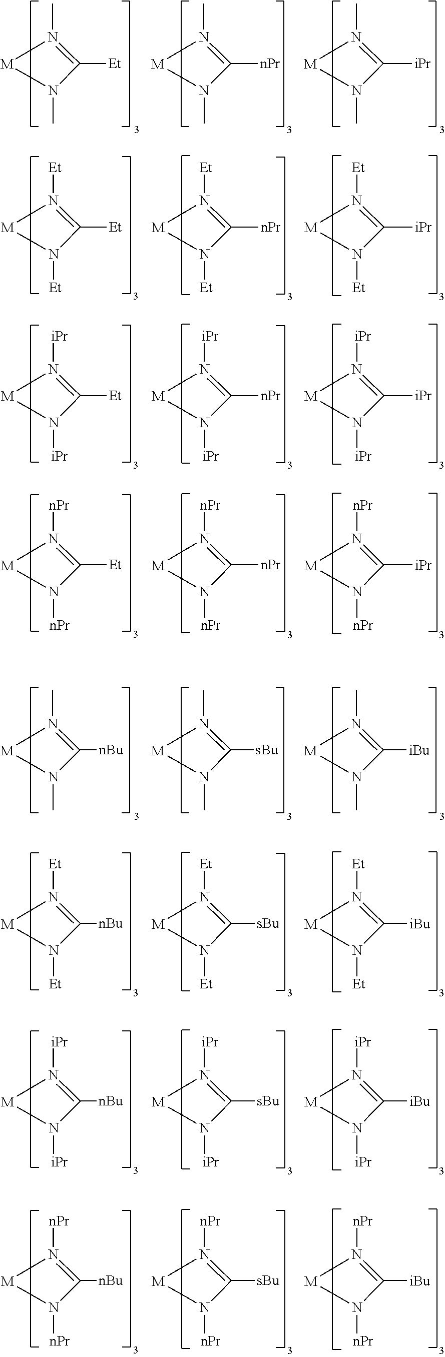

Additionally, when Lis an amine group, the following compounds may be exemplified.

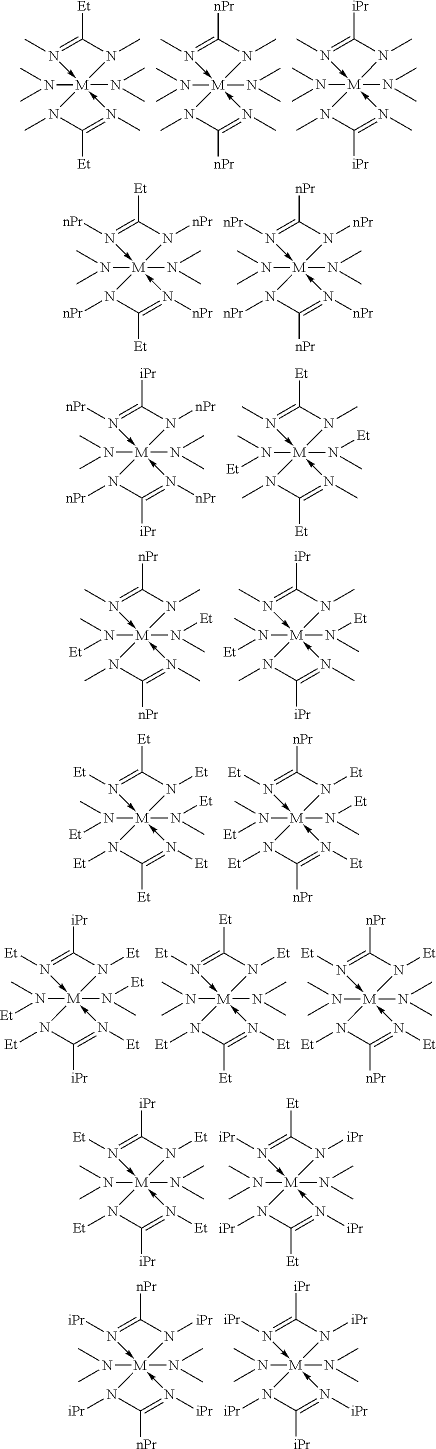

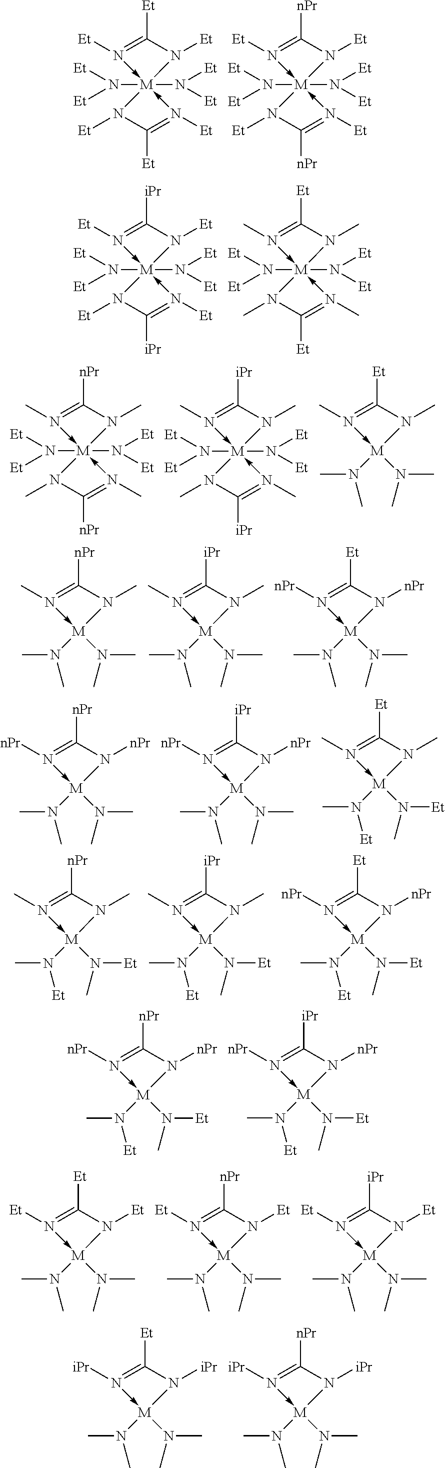

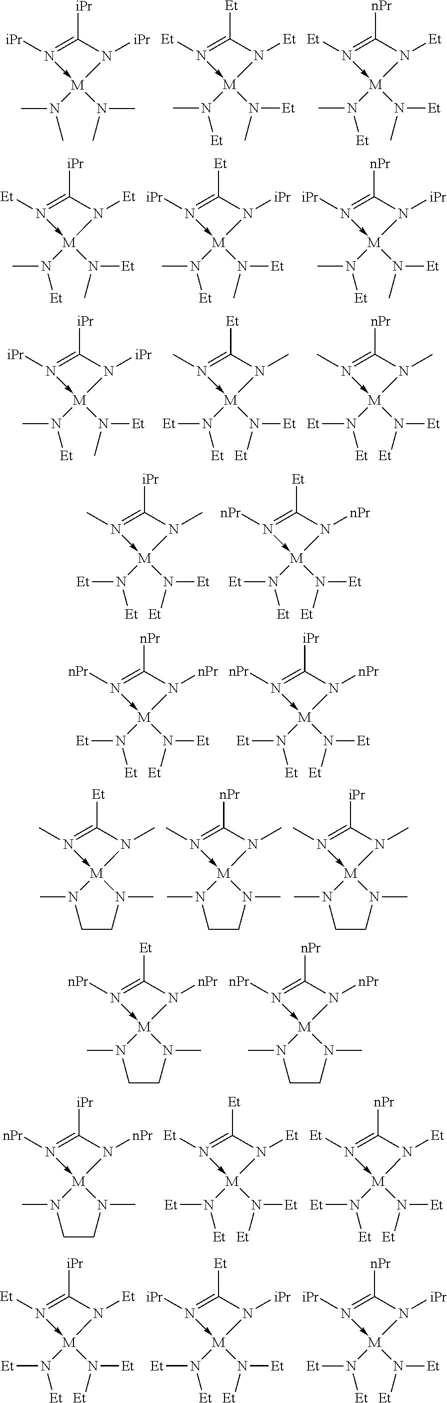

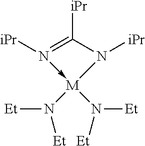

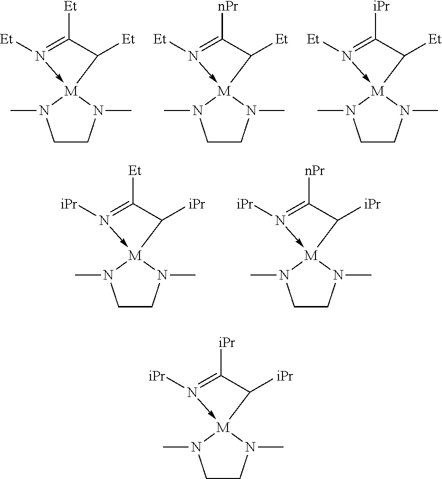

The precursor for forming a thin film, according to the present disclosure, contains an amidinate ligand, thus providing a precursor that exhibits low viscosity, high heat resistance, and high volatility, and is a liquid at room temperature, which enables the formation of a high-quality thin film. In particular, the precursor for forming a thin film has a viscosity of 60 cP or less (at 25° C.) and thus can exhibit properties suitable for use in the thin-film formation process.

Additionally, the precursor for forming a thin film of the present disclosure may further include a solvent for dissolving or diluting the precursor compound, in consideration of conditions and efficiency of the thin-film formation process. As for the solvent, any one or a mixture of saturated or unsaturated hydrocarbons having 1 to 16 carbon atoms, ketones, ethers, glymes, esters, tetrahydrofuran (THF), and tertiary amines may be used. Examples of the saturated or unsaturated hydrocarbons having 1 to 16 carbon atoms may include pentane, cyclohexane, ethylcyclohexane, heptane, octane, and toluene, and examples of the tertiary amines may include dimethylethylamine and triethylamine.

In particular, depending on the chemical structure, the precursor compound for forming a thin film may be in a solid form at room temperature. In this case, the compound can be dissolved by involving the solvent. In other words, when involving the solvent, the solvent has a content enabling dissolution of the precursor compound, which is preferably included in an amount in the range of 1 to 99 wt % with respect to the total weight of the precursor for forming a thin film.

The precursors with or without the solvent are vaporizable and thus may be fed into a chamber in the form of precursor gas. Therefore, depending on the types of precursor compounds, the precursor may be present as a liquid at room temperature and, when being easily vaporizable, the thin-film formation process may be performed even without involving additional solvents.

In this case, the thin-film formation process may be performed by any one of the following: a spin-on dielectric (SOD) process, a low-temperature plasma (LTP) process, a chemical vapor deposition (CVD) process, a plasma-enhanced CVD (PECVD) process, a high-density plasma CVD (HDPCVD) process, an atomic layer deposition (ALD) process, or a plasma-enhanced ALD (PEALD) process.

For example, when being applied, the HDPCVD process may be performed at high vacuum and high power compared to an atmospheric-pressure CVD (APCVD), low-pressure CVD (LPCVD), or PECVD process, thereby enabling the formation of a thin film that is structurally dense and exhibits excellent mechanical properties.

To this end, a method for forming a thin film, according to the present disclosure, includes a process of forming a thin film on a substrate using the above precursor for forming a thin film.

Specifically, the process of forming the thin film on the substrate may include the following processes: forming a precursor thin film through deposition of the precursor for forming a thin film onto the surface of the substrate, and reacting the precursor thin film with a reactant.

Additionally, for the deposition of the precursor, a process of vaporizing the precursor for forming a thin film so as to transfer the resulting vapor of the precursor into a chamber may be included.

Furthermore, the process of forming the thin film on the substrate may include a process of feeding the precursor for forming a thin film onto the substrate and applying a plasma in the presence of the reactant, thereby forming a metal thin film, an oxide thin film, a nitride thin film, an oxynitride thin film, and the like.

The process of forming the thin film may be performed under an in-chamber pressure in the range of 0.1 to 1000 mTorr. Additionally, it is suitable that a source power for plasma formation in the chamber is in the range of 500 to 9,000 W, while a bias power is in the range of 0 to 5,000 W. Furthermore, the bias power may not be applied in some cases.

In addition, the process of forming the thin film on the substrate is preferably performed at a temperature in the range of 150° C. to 500° C.

Additionally, when feeding the precursor for forming a thin film, a second metal precursor may be introduced, as needed, in order to further improve the electrical properties of a metal film to be ultimately formed, that is, to improve the capacitance or reduce the leakage current value. As for the second metal precursor, a metal precursor containing one or more metals (M″) selected from magnesium (Mg), strontium (Sr), barium (Ba), lanthanide (Ln), titanium (Ti), zirconium (Zr), hafnium (Hf), niobium (Nb), tantalum (Ta), aluminum (Al), indium (In), silicon (Si), germanium (Ge), and tin (Sn) atoms may further be optionally fed. The second metal precursor may be an alkoxy-based compound or an alkylamide-based compound containing the metal described above. When the metal is Si, examples of the second metal precursor used may include SiH(N(CH₃)₂)₃, SiH₂(N(C₂H₅)₂)₂, SiH₂(NHtBu)₂, SiH₃(N(iPr)₂), Si(OC₄H₉)₄, Si(OC₂H₅)₄, Si(OCH₃)₄, and Si(OC(CH₃)₃)₄.

The feeding of the second metal precursor may be performed in the same manner as the feeding of the precursor for forming a thin film. Additionally, the second metal precursor may be fed onto the substrate for forming a thin film in combination with the precursor, or may be fed sequentially after the precursor is completely fed.

Before being fed into the reaction chamber, the precursor and, optionally, the second metal precursor described above are preferably maintained at a temperature in the range of 50° C. to 250° C. and are more preferably maintained at a temperature in the range of 100° C. to 200° C., so as to make contact with the substrate for forming a thin film.

Additionally, after the step of feeding the precursor and before feeding the reactant, a purge process using an inert gas, such as argon (Ar), nitrogen (N₂), or helium (He), in a reactor may be performed to help the precursor and, optionally, the second metal precursor move onto the substrate or allow for the internal pressure of the reactor suitable for deposition and also to remove impurities from the chamber to the outside. In this case, the purge process using the inert gas is preferably performed so that the internal pressure of the reactor is in the range of 1 to 5 Torr.

Additionally, as for the reactant, any one or a mixture of the following may be used: nitrogen (N₂), ammonia (NH₃), hydrazine (N₂H₄), nitrous oxide (N₂O), oxygen (O₂), water vapor (H₂O), ozone (O₃), hydrogen peroxide (H₂O₂), silane, hydrogen (H), and diborane (B₂H). When the thin-film formation process is performed in the presence of oxidizing gases such as water vapor, oxygen, and ozone, as described above, a magnesium oxide thin film may be formed. Alternatively, when the thin-film formation process is performed in the presence of reducing gases such as hydrogen, ammonia, hydrazine, and silane, a pure metal thin film or metal nitride thin film may be formed. Additionally, a metal oxynitride thin film may be formed by a mixture of the reactants.

Furthermore, a treatment process through heat treatment or light irradiation may be performed in addition to plasma treatment. This treatment process is configured to provide the heat energy required to deposit the precursor for forming a thin film, and may be performed by known methods. Preferably, to manufacture thin films having the desired physical state and composition at a sufficient growth rate, it is desirable that the above treatment process is performed so that the temperature of the substrate in the reactor is in the range of 100° C. to 1,000° C., preferably in the range of 250° C. to 400° C.

Additionally, even during the above treatment process, a purge process using an inert gas, such as argon (Ar), nitrogen (N₂), or helium (He), in a reactor may be performed to help the reactant move onto the substrate or allow for the internal pressure of the reactor suitable for deposition and also to remove impurities or byproducts from the reactor to the outside, as described above.

The treatment process including the feeding of the precursor for forming a thin film, the feeding of the reactant, and the feeding of the inert gas, as described above, is defined as one cycle, and by repeatedly performing one or more cycles, a thin film can be formed.

Additionally, with the application of the thin-film formation process above, various semiconductor devices including such a thin film may be manufactured.

The following examples are to describe the effects of the present disclosure.

### Examples 1: Synthesis of Diethyl-Ethylamidinate (Et₂Et-AMD)

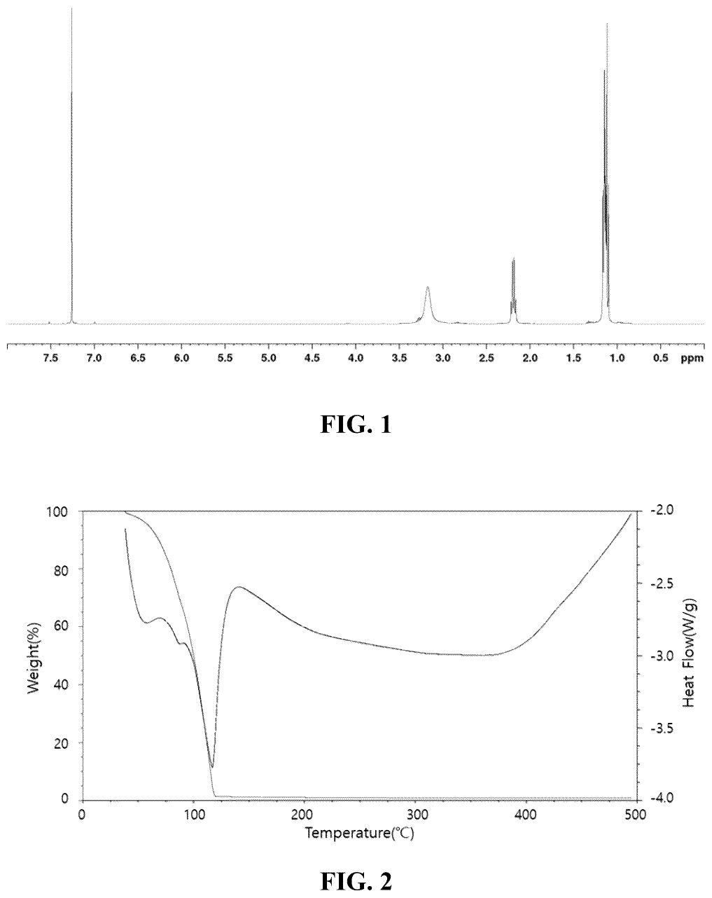

To a 500 mL Schlenk flask, 132.1 g (0.75 mol) of triethyl ortho-propionate and 74.3 g (1.65 mol) of ethylamine were added, followed by cooling to −30° C. and stirring. At the same temperature, 99.0 g (1.65 mol) of acetic acid was slowly added dropwise to the resulting mixture, which was then subjected to reflux at 170° C. The solvent was removed under reduced pressure after 18 hours, followed by extraction with a 5 N aqueous sodium hydroxide (NaOH) solution and diethyl ether. The extracts were dried over magnesium sulfate and then filtered, followed by removal of the solvent under reduced pressure. The residues were distilled under reduced pressure, thus obtaining 46.1 g (48%) of a colorless liquid compound. The NMR result is as shown in FIG. 1.

¹H NMR (CDCl₃, 25° C.): 1.13 (m, 9H), 2.20 (q, 2H), 3.17 (m, 4H).

Purification conditions: at 75° C. to 78° C. and 2.4 Torr

During TGA measurement performed at a temperature ramp rate of 10° C./min in a nitrogen atmosphere at a flow rate of 200 mL/min, the pale yellow liquid had a residual mass of 0%, indicating that there were almost no residues left. This result was confirmed by the TGA result (FIG. 2), which shows the percentage of weight loss as a function of temperature.

### Example 2: Synthesis of diethyl-n-propylamidinate (Et₂nPr-AMD)

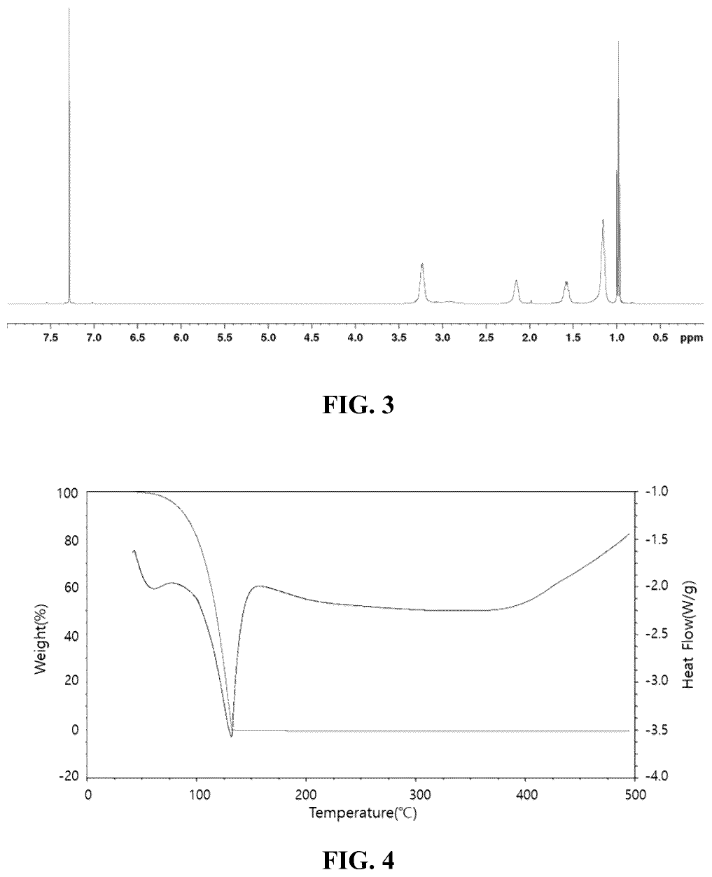

To a 500 mL Schlenk flask, 122.5 g (0.64 mol) of 1,1,1-triethoxybutane and 58.0 g (1.29 mol) of ethylamine were added, followed by cooling to −30° C. and stirring. At the same temperature, 77.3 g (1.29 mol) of acetic acid was slowly added dropwise to the resulting mixture, which was then subjected to reflux at 170° C. The solvent was removed under reduced pressure after 18 hours, followed by extraction with a 5 N aqueous sodium hydroxide (NaOH) solution and diethyl ether. The extracts were dried over magnesium sulfate and then filtered, followed by removal of the solvent under reduced pressure. The residues were distilled under reduced pressure, thus obtaining 36.6 g (40%) of a colorless liquid compound. The NMR result is as shown in FIG. 3.

¹H NMR (CDCl₃, 25° C.): 0.98 (t, 3H), 1.15 (m, 6H), 1.59 (m, 2H), 2.15 (m, 2H), 3.22 (m, 4H).

Purification conditions: at 82° C. to 87° C. and 0.5 Torr

During TGA measurement performed at a temperature ramp rate of 10° C./min in a nitrogen atmosphere at a flow rate of 200 mL/min, the pale yellow liquid had a residual mass of 0%, indicating that there were almost no residues left. This result was confirmed by the TGA result (FIG. 4), which shows the percentage of weight loss as a function of temperature.

### Example 3: Synthesis of di-n-propyl-ethylamidinate (nPr₂Et-AMD)

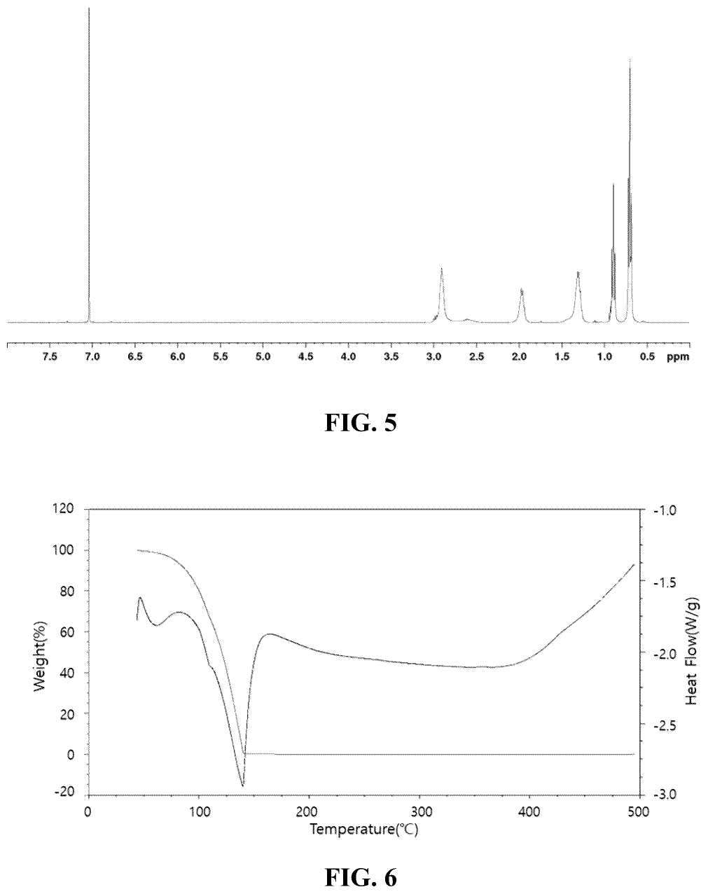

To a 500 mL Schlenk flask, 132.1 g (0.75 mol) of triethyl ortho-propionate and 97.5 g (1.65 mol) of n-propylamine were added, followed by cooling to −30° C. and stirring. At the same temperature, 49.5 g (0.83 mol) of acetic acid was slowly added dropwise to the resulting mixture, which was then subjected to reflux at 170° C. The solvent was removed under reduced pressure after 18 hours, followed by extraction with a 5 N aqueous sodium hydroxide (NaOH) solution and diethyl ether. The extracts were dried over magnesium sulfate and then filtered, followed by removal of the solvent under reduced pressure. The residues were distilled under reduced pressure, thus obtaining 48.0 g (41%) of a colorless liquid compound. The NMR result is as shown in FIG. 5.

¹H NMR (CDCl₃, 25° C.): 0.70 (t, 6H), 0.89 (t, 3H), 1.31 (m, 4H), 1.97 (m, 2H), 2.90 (m, 4H).

Purification conditions: at 80° C. to 90° C. and 0.5 Torr

During TGA measurement performed at a temperature ramp rate of 10° C./min in a nitrogen atmosphere at a flow rate of 200 mL/min, the pale yellow liquid had a residual mass of 0%, indicating that there were almost no residues left. This result was confirmed by the TGA result (FIG. 6), which shows the percentage of weight loss as a function of temperature.

### Example 4: Synthesis of bis(ethylcyclopentadienyl)(diethyl-ethylamidinato)yttrium [(EtCp)₂Y(Et₂Et-AMD)]

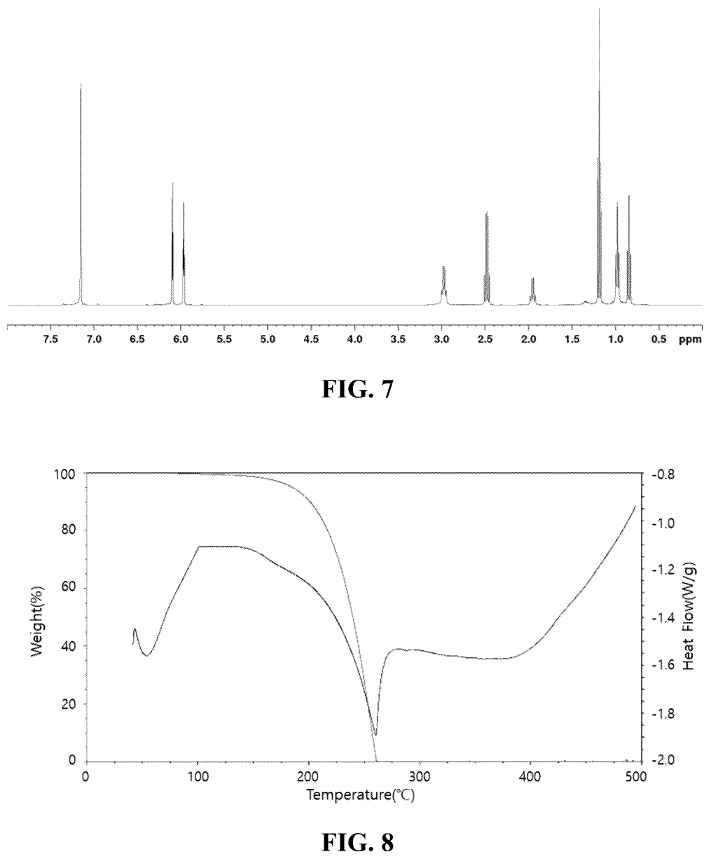

To 300 mL of THF, 32.83 g (0.256 mol) of diethyl-ethylamidinate was added, followed by cooling to −78° C., and 102.4 mL (0.256 mol) of an n-BuLi hexane solution (2.5 M) was then slowly added dropwise, thus preparing Li—(Et₂Et-AMD). The resulting solution was stirred at −78° C. for 30 minutes, heated to room temperature, and then stirred for an additional 2 hours at room temperature. The prepared Li—(Et₂Et-AMD) solution was slowly added dropwise to a flask containing (EtCp)₂YCl at room temperature and then stirred at room temperature for 12 hours. The resulting mixture was evaporated under vacuum, followed by dissolution in 250 mL of pentane, filtration, and evaporation of the solvent and volatiles under vacuum to produce a red liquid, which was then treated at 165° C. and 40 mTorr, thus obtaining a pale yellow liquid. The yield was 60.1 g (58.3%). The NMR result is as shown in FIG. 7.

¹H NMR (C₆D₆, 25° C.): 0.85 (t, 3H), 0.98 (t, 6H), 1.20 (t, 6H), 1.97 (q, 2H), 2.48 (q, 4H), 2.97 (q, 4H), 6.0 (dt, 8H).

During TGA measurement performed at a temperature ramp rate of 10° C./min in a nitrogen atmosphere at a flow rate of 200 mL/min, the pale yellow liquid had a residual mass of 0%, indicating that there were almost no residues left. This result was confirmed by the TGA result (FIG. 8), which shows the percentage of weight loss as a function of temperature.

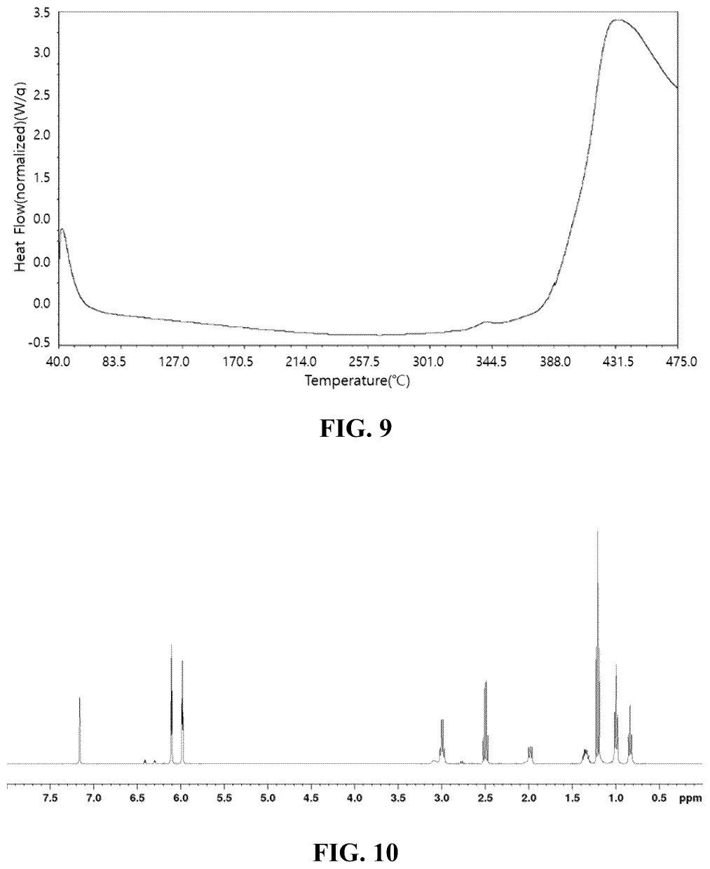

The pale yellow liquid sample was placed in a sealed container for DSC and maintained at 40° C. for 10 minutes. Subsequently, a decomposition peak was observed at 377° C. during DSC measurement (Discovery 25 from TA Instruments) performed at a temperature ramp rate of 10° C./min. This result was confirmed by the DSC result (FIG. 9), which shows the heat energy change as a function of temperature.

The pale yellow liquid sample was placed in a rotational viscosity measurement container for viscosity measurement at 25° C. using a low-viscosity spindle. A viscosity of 30 cP was measured during viscosity measurement at 12 rpm.

### Example 5: Synthesis of bis(ethylcyclopentadienyl)(diethyl-n-propylamidinato)yttrium [(EtCp)₂Y(Et₂nPr-AMD)]

To 300 mL of THF, 36.43 g (0.256 mol) of diethyl-n-propylamidinate was added, followed by cooling to −78° C., and 102.4 mL (0.256 mol) of an n-BuLi hexane solution (2.5 M) was then slowly added dropwise, thus preparing Li—(Et₂nPr-AMD). The resulting solution was stirred at −78° C. for 30 minutes, heated to room temperature, and then stirred for an additional 2 hours at room temperature. The prepared Li—(Et₂nPr-AMD) solution was slowly added dropwise to a flask containing Y(EtCp)₂Cl at room temperature and then stirred at room temperature for 12 hours. The resulting mixture was evaporated under vacuum, followed by dissolution in 250 mL of pentane, filtration, and evaporation of the solvent and volatiles under vacuum to produce a red liquid, which was then distilled at 185° C. and 40 mTorr, thus obtaining a pale yellow liquid. The yield was 65.1 g (61.0%). The NMR result is as shown in FIG. 10.

¹H NMR (C₆D₆, 25° C.): 0.83 (t, 3H), 0.99 (t, 6H), 1.20 (t, 6H), 1.36 (q, 2H), 1.98 (q, 2H), 2.50 (q, 4H), 3.00 (q, 4H), 6.0 (dt, 8H).

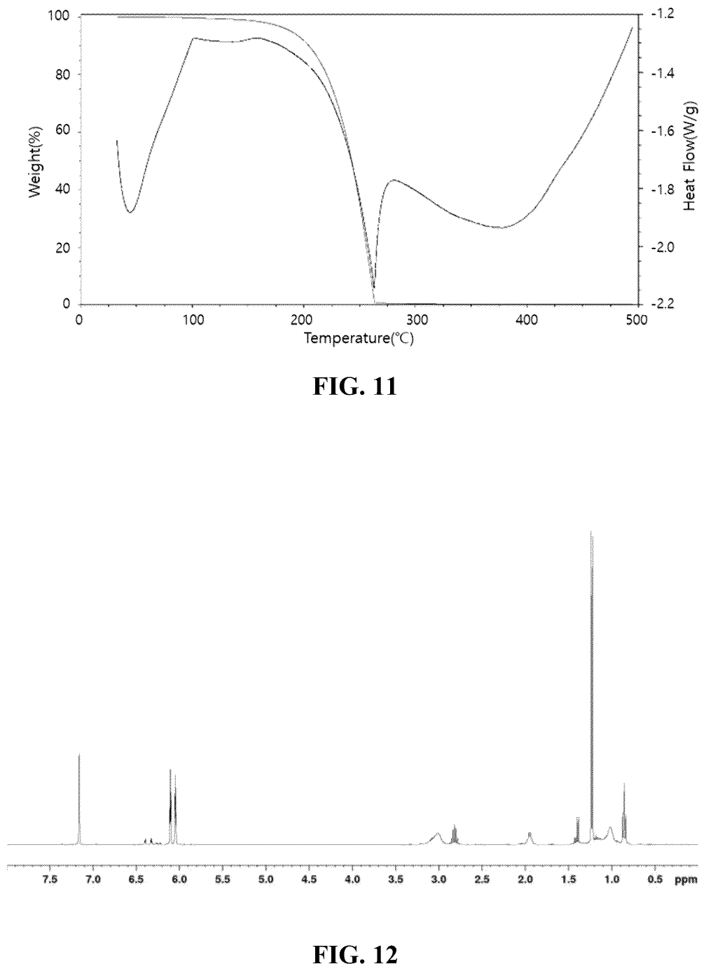

During TGA measurement performed at a temperature ramp rate of 10° C./min in a nitrogen atmosphere at a flow rate of 200 mL/min, the pale yellow liquid had a residual mass of 0%, indicating that there were almost no residues left. This result was confirmed by the TGA result (FIG. 11), which shows the percentage of weight loss as a function of temperature.

The pale yellow liquid sample was placed in a rotational viscosity measurement container for viscosity measurement at 25° C. using a low-viscosity spindle. A viscosity of 28 cP was measured during viscosity measurement at 20 rpm.

### Example 6: Synthesis of bis(isopropylcyclopentadienyl)(diethyl-ethylamidinato)yttrium [(iPrCp)₂Y(Et₂Et-AMD)]

To 300 mL of THF, 32.83 g (0.256 mol) of diethyl-ethylamidinate was added, followed by cooling to −78° C., and 102.4 mL (0.256 mol) of an n-BuLi hexane solution (2.5 M) was then slowly added dropwise, thus preparing Li—(Et₂Et-AMD). The resulting solution was stirred at −78° C. for 30 minutes, heated to room temperature, and then stirred for an additional 2 hours at room temperature. The prepared Li—(Et₂Et-AMD) solution was slowly added dropwise to a flask containing (iPrCp)₂YCl at room temperature and then stirred at room temperature for 12 hours. The resulting mixture was evaporated under vacuum, followed by dissolution in 250 mL of pentane, filtration, and evaporation of the solvent and volatiles under vacuum to produce a red liquid, which was then distilled at 170° C. and 20 mTorr, thus obtaining a yellow liquid. The yield was 63.6 g (70.1%). The ¹H NMR (AV400 MHz HD from Bruker) result is illustrated in FIG. 12.

¹H NMR (C₆D₆, 25° C.): 0.85 (t, 3H), 1.02 (t, 6H), 1.24 (d, 12H), 1.94 (q, 2H), 2.82 (m, 2H), 3.01 (q, 4H), 6.01 (dt, 8H).

During TGA measurement (SDT Q600 from TA Instruments) performed at a temperature ramp rate of 10° C./min in a nitrogen atmosphere at a flow rate of 200 mL/min, the yellow liquid had a residual mass of 0%, indicating that there were almost no residues left.

### Example 7: Synthesis of bis(methylcyclopentadienyl)(diethyl-n-propylamidinato)yttrium [(MeCp)₂Y(Et₂nPr-AMD)]

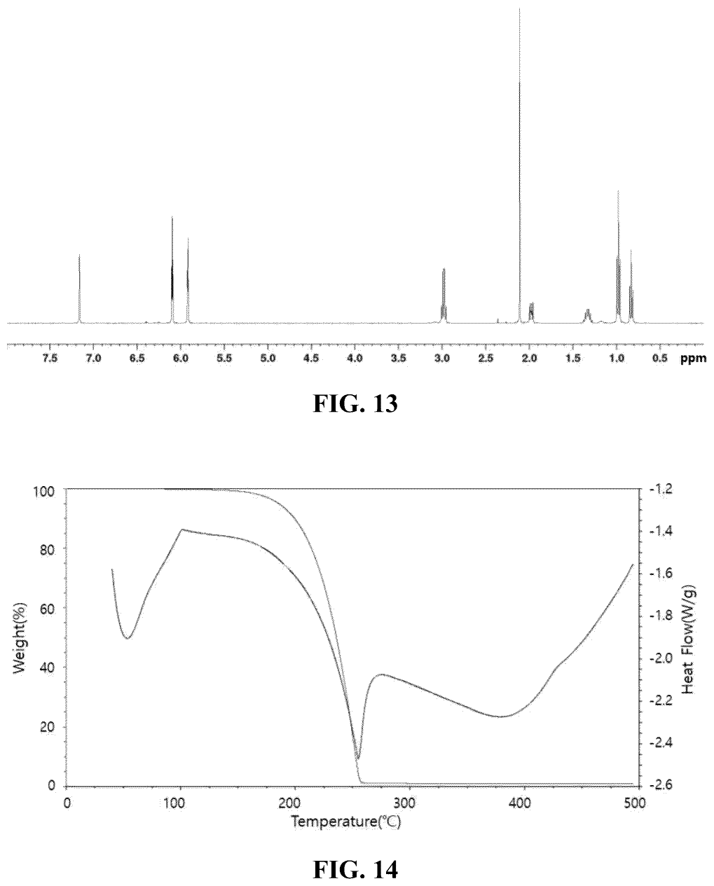

To 300 mL of THF, 36.43 g (0.256 mol) of diethyl-n-propylamidinate was added, followed by cooling to −78° C., and 102.4 mL (0.256 mol) of an n-BuLi hexane solution (2.5 M) was then slowly added dropwise, thus preparing Li—(Et₂nPr-AMD). The resulting solution was stirred at −78° C. for 30 minutes, heated to room temperature, and then stirred for an additional 2 hours at room temperature. The prepared Li—(Et₂nPr-AMD) solution was slowly added dropwise to a flask containing (MeCp)₂YCl at room temperature and then stirred at room temperature for 12 hours. The resulting mixture was evaporated under vacuum, followed by dissolution in 250 mL of pentane, filtration, and evaporation of the solvent and volatiles under vacuum to produce a red liquid, which was then treated at 170° C. and 67 mTorr, thus obtaining a colorless liquid. The yield was 59.11 g (59.4%). The ¹H NMR (AV400 MHz HD from Bruker) result is illustrated in FIG. 13.

¹H NMR (C₆D₆, 25° C.): 0.83 (t, 3H), 0.98 (t, 6H), 1.34 (t, 2H), 1.96 (q, 2H), 2.11 (s, 6H), 2.97 (q, 4H), 6.0 (dt, 8H).

During TGA measurement (SDT Q600 from TA Instruments) performed at a temperature ramp rate of 10° C./min in a nitrogen atmosphere at a flow rate of 200 mL/min, the colorless liquid had a residual mass of 0.9%, indicating that there were almost no residues left. This result is illustrated in FIG. 14, which shows the TGA result indicating the percentage of weight loss as a function of temperature.

The colorless liquid sample was placed in a sealed container for DSC and maintained at 40° C. for 10 minutes. Subsequently, a decomposition peak was observed at 410° C. during DSC measurement performed at a temperature ramp rate of 10° C./min.

### Example 8: Synthesis of bis(methylcyclopentadienyl)(di-n-propyl-ethylamidinato)yttrium [(MeCp)₂Y(nPr₂Et-AMD)]

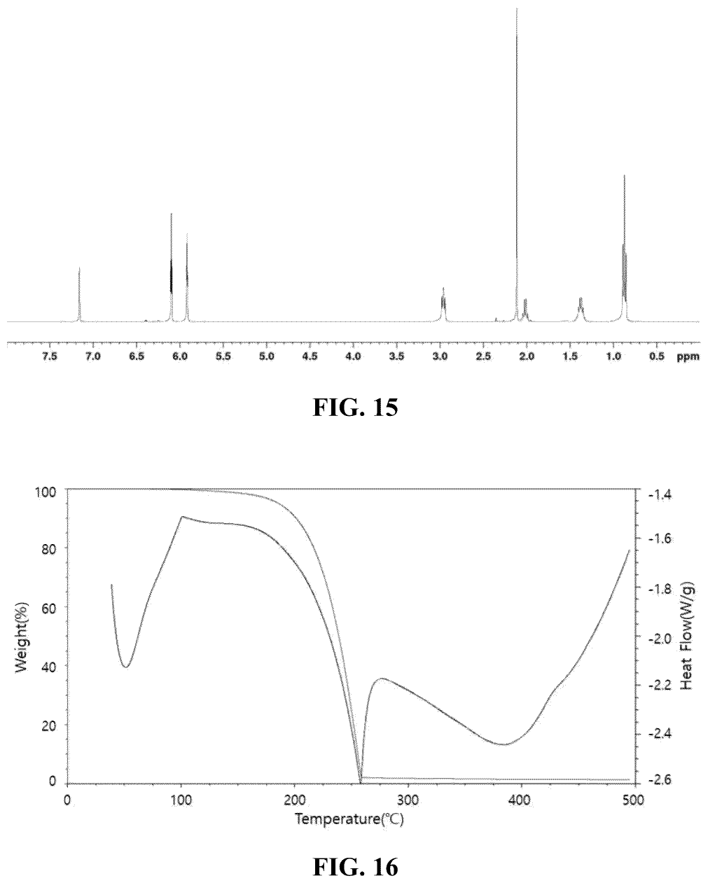

To 300 mL of THF, 40.02 g (0.256 mol) of di-n-propyl-ethylamidinate was added, followed by cooling to −78° C., and 102.4 mL (0.256 mol) of an n-BuLi hexane solution (2.5 M) was then slowly added dropwise, thus preparing Li-(nPr₂Et-AMD). The resulting solution was stirred at −78° C. for 30 minutes, heated to room temperature, and then stirred for an additional 2 hours at room temperature. The prepared Li-(nPr₂Et-AMD) solution was slowly added dropwise to a flask containing (MeCp)₂YCl at room temperature and then stirred at room temperature for 12 hours. The resulting mixture was evaporated under vacuum, followed by dissolution in 250 mL of pentane, filtration, and evaporation of the solvent and volatiles under vacuum to produce a red liquid, which was then treated at 155° C. and 113 mTorr, thus obtaining a yellow liquid. The yield was 60.1 g (58.3%). The NMR (AV400 MHz HD from Bruker) result is illustrated in FIG. 15.

¹H NMR (C₆D₆, 25° C.): 0.87 (t, 9H), 1.37 (q, 4H), 2.01 (q, 2H), 2.12 (s, 6H), 2.96 (q, 4H), 6.0 (dt, 8H).

During TGA measurement (SDT Q600 from TA Instruments) performed at a temperature ramp rate of 10° C./min in a nitrogen atmosphere at a flow rate of 200 mL/min, the yellow liquid had a residual mass of 1.6%, indicating that there were almost no residues left. This result is illustrated in FIG. 16, which shows the TGA result indicating the percentage of weight loss as a function of temperature.

### Example 9: Synthesis of bis(ethylcyclopentadienyl)(dimethyl-propylamidinato)yttrium [(EtCp)₂Y(Me₂-nPrAMD)]

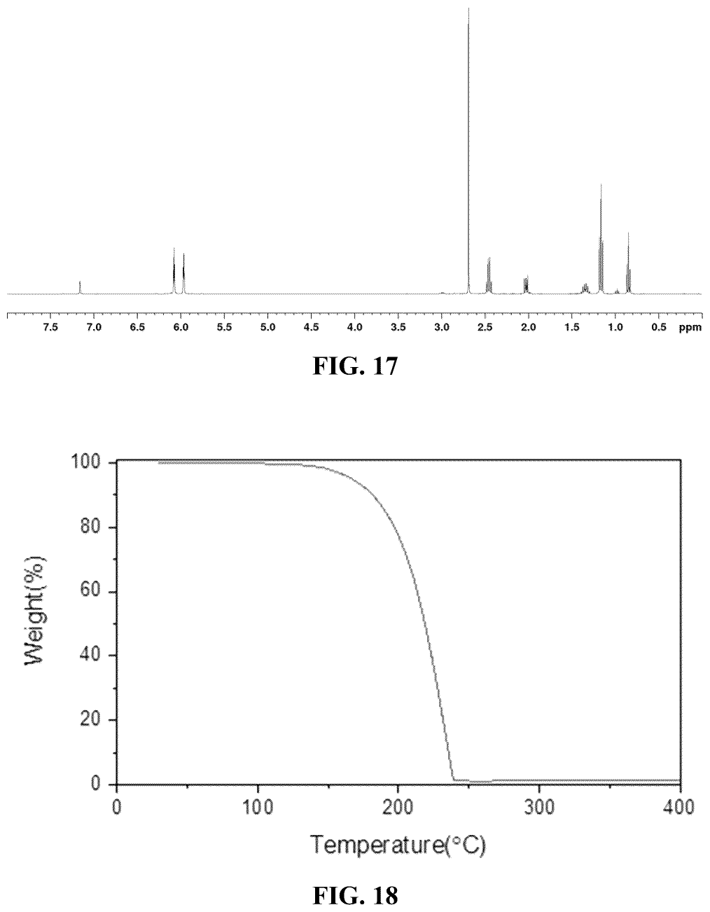

To 300 mL of THF, 29.24 g (0.256 mol) of dimethyl-n-propylamidinate was added, followed by cooling to −78° C., and 102.4 mL (0.256 mol) of an n-BuLi hexane solution (2.5 M) was then slowly added dropwise, thus preparing Li—(Me₂-nPrAMD). The resulting solution was stirred at −78° C. for 30 minutes, heated to room temperature, and then stirred for an additional 2 hours at room temperature. The prepared Li—(Me₂-nPrAMD) solution was slowly added dropwise to a flask containing (EtCp)₂YCl at room temperature and then stirred at room temperature for 12 hours. The resulting mixture was evaporated under vacuum, followed by dissolution in 250 mL of pentane, filtration, and evaporation of the solvent and volatiles under vacuum to produce a pale pink liquid, which was then treated at 152° C. and 44 mTorr, thus obtaining an orange liquid. The yield was 85.0 g (85.5%). The ¹H NMR (AV400 MHz HD from Bruker) result is illustrated in FIG. 17.

¹HNMR (C₆D₆, 25° C.): 0.85 (t, 3H), 1.16 (t, 6H), 1.33 (m, 2H), 2.00 (m, 2H), 2.45 (q, 4H), 2.69 (s, 6H), 6.0 (dt, 8H).

During TGA measurement (SDT Q600 from TA Instruments) performed at a temperature ramp rate of 10° C./min in a nitrogen atmosphere at a flow rate of 200 mL/min, the orange liquid had a residual mass of 1%, indicating that there were almost no residues left. This result is illustrated in FIG. 18, which shows the TGA result indicating the percentage of weight loss as a function of temperature.

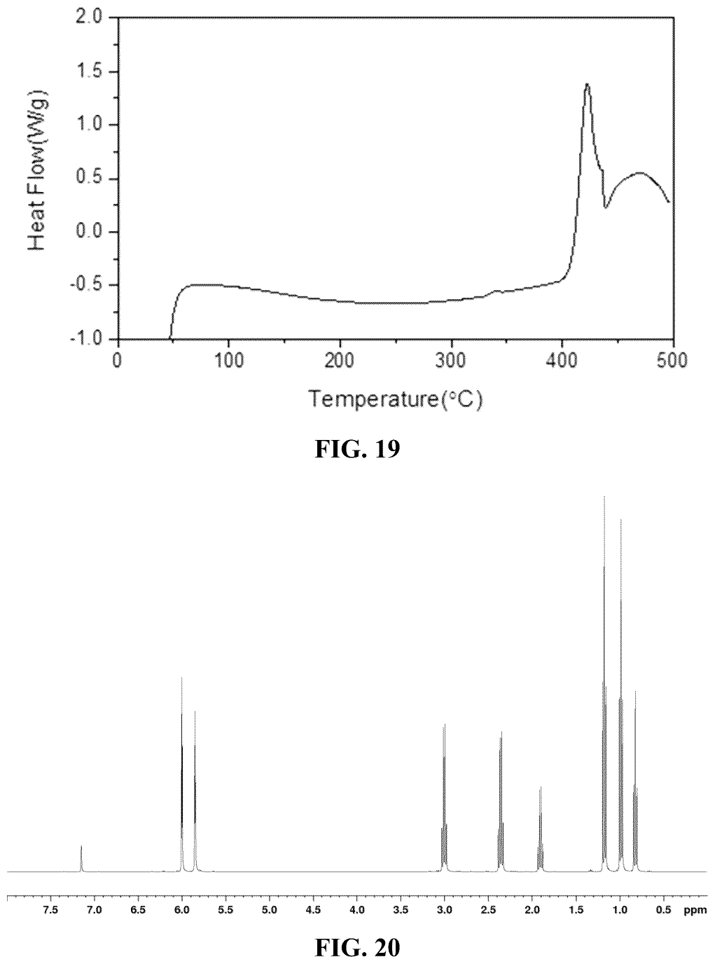

The orange liquid sample was placed in a sealed container for DSC and maintained at 40° C. for 10 minutes. Subsequently, a decomposition peak was observed at 422° C. during DSC measurement (Discovery 25 from TA instruments) performed at a temperature ramp rate of 10° C./min. This result is illustrated in FIG. 19, which shows the DSC result indicating the heat energy change as a function of temperature.

### Example 10: Synthesis of bis(ethylcyclopentadienyl)(diethyl-n-propylamidinato)scandium [(EtCp)₂Sc(Et₂nPr-AMD)]

To 300 mL of THF, 47.01 g (0.330 mol) of diethyl-n-propylamidinate was added, followed by cooling to −78° C., and 132.2 mL (0.330 mol) of an n-BuLi hexane solution (2.5 M) was then slowly added dropwise, thus preparing Li—(Et₂nPr-AMD). The resulting solution was stirred at −78° C. for 30 minutes, heated to room temperature, and then stirred for an additional 2 hours at room temperature. The prepared Li—(Et₂nPr-AMD) solution was slowly added dropwise to a flask containing (EtCp)₂ScCl at room temperature and then stirred at room temperature for 12 hours. The resulting mixture was evaporated under vacuum, followed by dissolution in 250 mL of pentane, filtration, and evaporation of the solvent and volatiles under vacuum to produce a red liquid, which was then distilled at 150° C. and 28.4 mTorr, thus obtaining a colorless liquid. The yield was 73.33 g (59.6%). The ¹H NMR (AV400 MHz HD from Bruker) result is illustrated in FIG. 20.

¹H NMR (C₆D₆, 25° C.): 0.82 (t, 3H), 0.98 (t, 6H), 1.17 (t, 6H), 1.91 (q, 2H), 2.38 (q, 4H), 3.01 (q, 4H), 6.0 (dt, 8H).

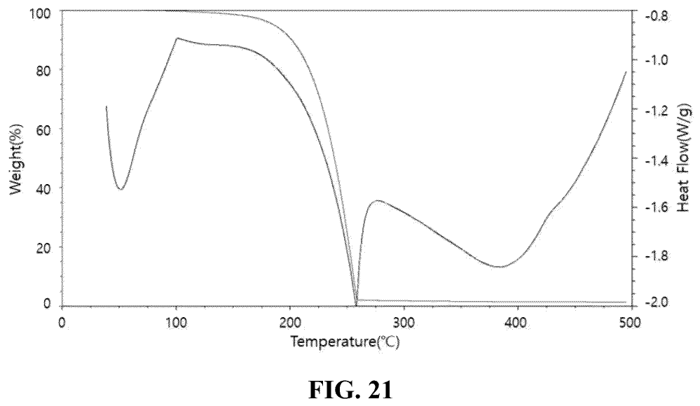

During TGA measurement performed at a temperature ramp rate of 10° C./min in a nitrogen atmosphere at a flow rate of 200 mL/min, the colorless liquid sample had a residual mass of 1.2%. This result is illustrated in FIG. 21, which shows the TGA result indicating the percentage of weight loss as a function of temperature.

### Example 11: Synthesis of bis(isopropylcyclopentadienyl)(diethyl-n-propylamidinato)cerium [(iPrCp)₂Ce(Et₂nPr-AMD)]

To a 500 mL Schlenk flask, 6.13 g (0.256 mol) of NaH and 140 mL of THF were added, followed by slowly adding 11.54 g (0.081 mol) of diethyl-n-propylamidinate and 17.56 g (0.162 mol) of isopropylcyclopentadiene dropwise at 0° C. The resulting mixture was stirred at room temperature for 12 hours, thus preparing a mixture solution of Na-EtCp and Na—(Et₂Et-AMD). The Schlenk flask containing the reactants was cooled to −10° C., followed by slowly adding 20 g (0.081 mol) of cerium chloride. The resulting solution was stirred at room temperature for 12 hours and then evaporated under vacuum to obtain a liquid, which was then purified by distillation at 250° C. and 50 mTorr, thus obtaining a dark purple liquid. The yield was 29.5 g (73.4%).

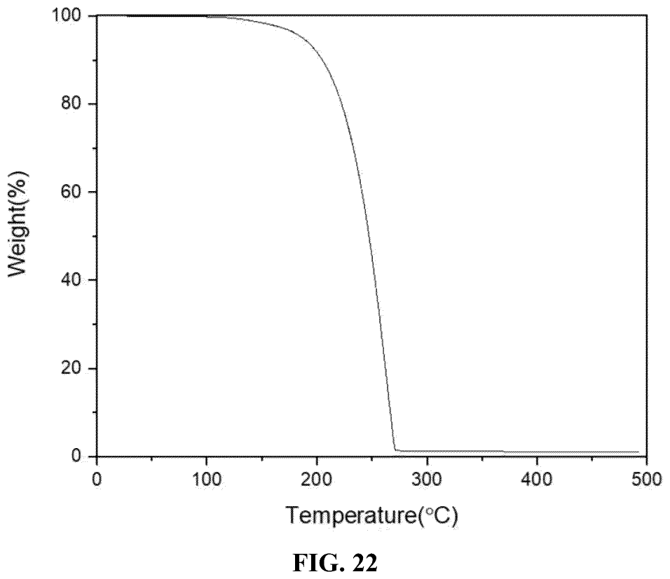

During TGA measurement (SDT Q600 from TA Instruments) performed at a temperature ramp rate of 10° C./min in a nitrogen atmosphere at a flow rate of 200 mL/min, the dark purple liquid had a T_{1/2} value of 247.4° C. and a residual mass of 1.1% at 350° C., indicating that there were almost no residues left. This result is illustrated in FIG. 22, which shows the TGA result indicating the percentage of weight loss as a function of temperature.

It was confirmed through the TGA result showing a single volatilization curve that the target compound was synthesized.

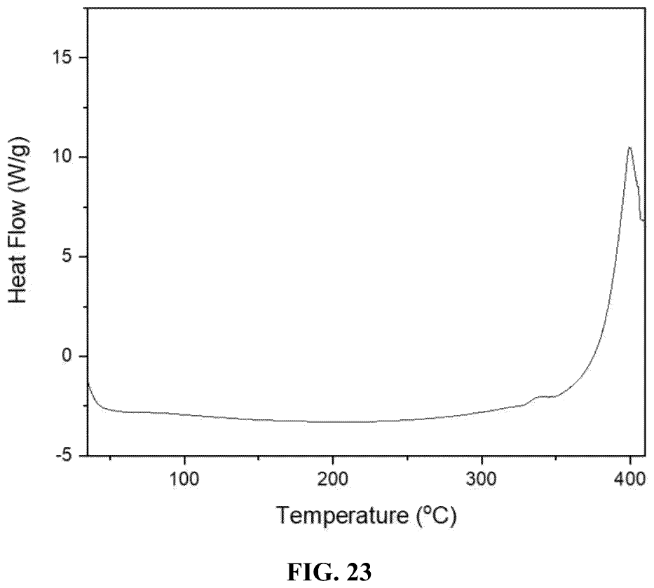

Additionally, the dark purple liquid sample was placed in a sealed container for DSC and maintained at 40° C. for 10 minutes. Subsequently, a decomposition peak was observed at 399° C. during DSC measurement (Discovery 25 from TA Instruments) performed at a temperature ramp rate of 10° C./min. This result is illustrated in FIG. 23, which shows the DSC result indicating the heat energy change as a function of temperature.

### Example 12: Synthesis of bis(ethylcyclopentadienyl)(diethyl-ethylamidinato)gadolinium [(EtCp)₂Gd(Et₂Et-AMD)]

To a 500 mL Schlenk flask, 6.13 g (0.256 mol) of NaH and 140 mL of THF were added, followed by slowly adding 11.54 g (0.081 mol) of diethyl-n-propylamidinate and 17.56 g (0.162 mol) of isopropylcyclopentadiene dropwise at 0° C. The resulting mixture was stirred at room temperature for 12 hours, thus preparing a mixture solution of Na-EtCp and Na—(Et₂Et-AMD). The Schlenk flask containing the reactants was cooled to −10° C., followed by slowly adding 20 g (0.081 mol) of gadolinium chloride. The resulting solution was stirred at room temperature for 12 hours and then evaporated under vacuum to obtain a liquid, which was then purified by distillation at 250° C. and 50 mTorr, thus obtaining a pale yellow liquid. The yield was 29.5 g (73.4%).

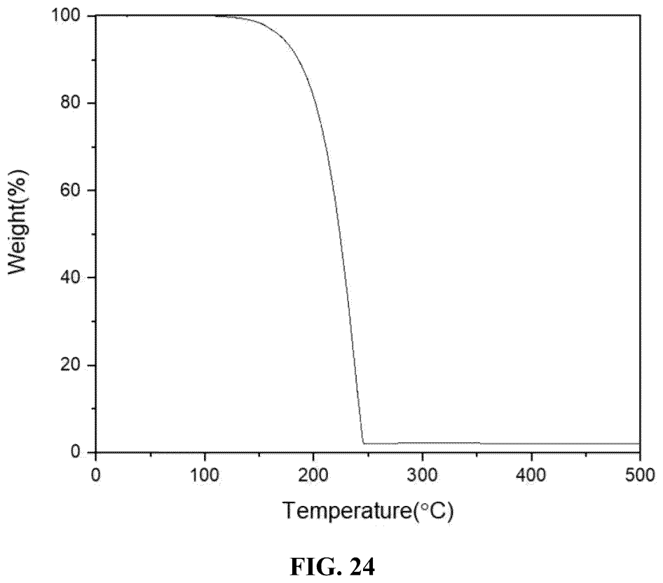

During TGA measurement (SDT Q600 from TA Instruments) performed at a temperature ramp rate of 10° C./min in a nitrogen atmosphere at a flow rate of 200 m/min, the pale yellow liquid was found to have a T_{1/2} value of 224° C. and a residual mass of 2% at 350° C. This result is illustrated in FIG. 24, which shows the TGA result indicating the percentage of weight loss as a function of temperature.

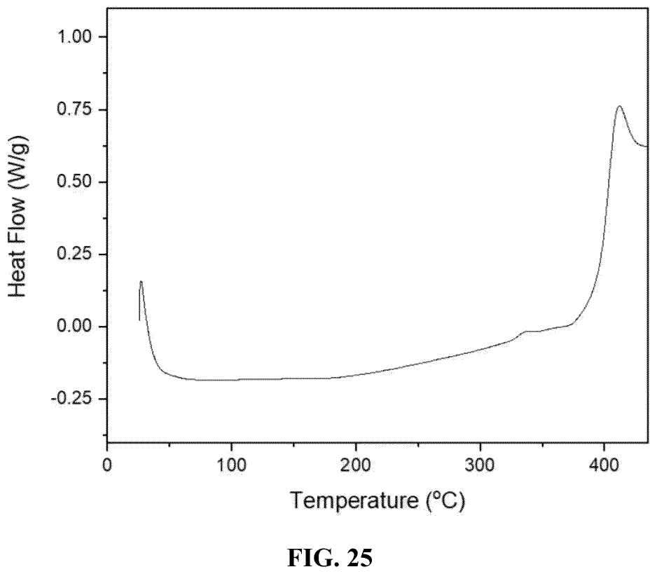

It was confirmed through the TGA result showing a single volatilization curve that the target compound was synthesized. Additionally, the pale yellow liquid sample was placed in a sealed container for DSC and maintained at 40° C. for 10 minutes. Subsequently, a decomposition peak was observed at 412° C. during DSC measurement (Discovery 25 from TA Instruments) performed at a temperature ramp rate of 10° C./min. This result is illustrated in FIG. 25, which shows the DSC result indicating the heat energy change as a function of temperature.

Furthermore, the pale yellow liquid was analyzed using a viscometer (DV2T viscometer from Brookfield Ametek) in a nitrogen atmosphere for viscosity measurement, and was confirmed to exhibit a low viscosity of 31 cP at 25° C.

### Example 13: Synthesis of bis(ethylcyclopentadienyl)(diethyl-ethylamidinato)dysprosium [(EtCp)₂Dy(Et₂Et-AMD)]

To a 500 ml Schlenk flask, 160 ml of THF, 9.54 g (0.0744 mol) of diethyl-ethylamidinate, and 14.29 g (0.1489 mol) of ethylcyclopentadiene were added, followed by cooling to 0° C. To the cooled solution, 89.33 mL (0.2233 mol) of an n-BuLi hexane solution (2.5 M) was slowly added dropwise, followed by stirring at room temperature for 1 hour, thus preparing a mixture solution of Li-EtCp and Li—(Et₂Et-AMD). The Schlenk flask containing the reactants was cooled to 0° C., and 20 g (0.0744 mol) of dysprosium chloride was added. The resulting solution was stirred at room temperature for 3 hours and then evaporated under vacuum to obtain a pale green liquid, which was then purified by distillation at 170° C. and 58 mTorr, thus obtaining a pale green liquid. The yield was 19.7 g (55.6%).

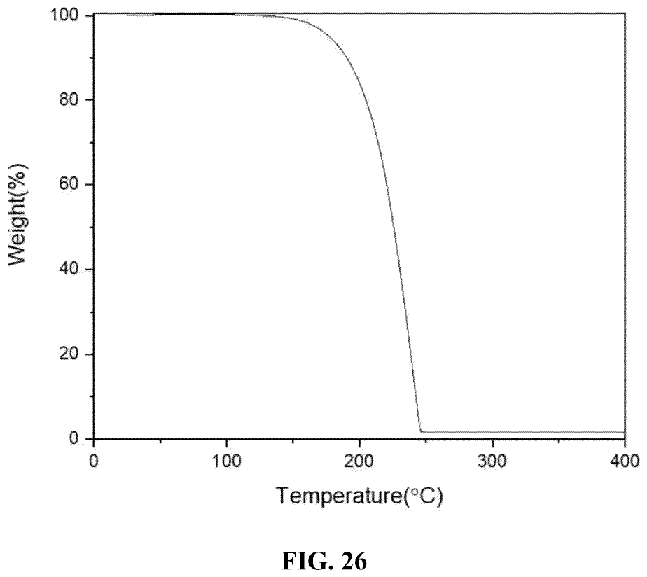

During TGA measurement (SDT Q600 from TA Instruments) performed at a temperature ramp rate of 10° C./min in a nitrogen atmosphere at a flow rate of 200 mL/min, the pale green liquid had a T_{1/2} value of 225.8° C. and a residual mass of 1.58% at 350° C., indicating that there were almost no residues left. This result is illustrated in FIG. 26, which shows the TGA result indicating the percentage of weight loss as a function of temperature.

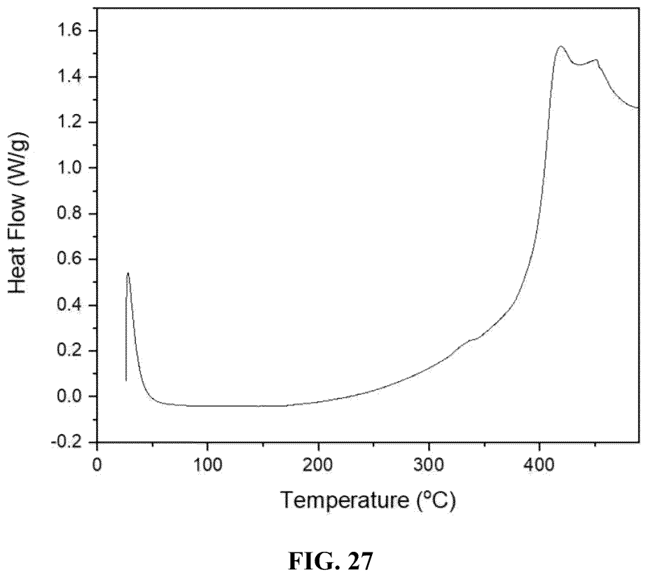

It was confirmed through the TGA result showing a single volatilization curve that the target compound was synthesized. Additionally, the pale green liquid sample was placed in a sealed container for DSC and maintained at 40° C. for 10 minutes. Subsequently, a decomposition peak was observed at 418° C. during DSC measurement (Discovery 25 from TA Instruments) performed at a temperature ramp rate of 10° C./min. This result is illustrated in FIG. 27, which shows the DSC result indicating the heat energy change as a function of temperature.

Furthermore, the pale green liquid was analyzed using a viscometer (DV2T viscometer from Brookfield Ametek) in a nitrogen atmosphere for viscosity measurement, and was confirmed to exhibit a low viscosity of 38 cP at 25° C.

### Example 14: Synthesis of bis(ethylcyclopentadienyl)(diethyl-ethylamidinato)lutetium [(EtCp)₂Lu(Et₂Et-AMD)]

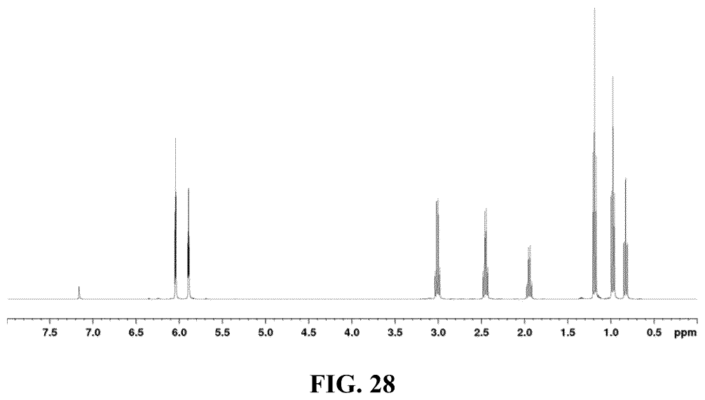

To a 500 ml Schlenk flask, 160 ml of THF, 9.12 g (0.0711 mol) of diethyl-ethylamidinate, and 13.39 g (0.1422 mol) of ethylcyclopentadiene were added, followed by cooling to 0° C. To the cooled solution, 85.31 mL (0.2133 mol) of an n-BuLi hexane solution (2.5 M) was slowly added dropwise, followed by stirring at room temperature for 1 hour, thus preparing a mixture solution of Li-EtCp and Li—(Et₂Et-AMD). The Schlenk flask containing the reactants was cooled to 0° C., and 20 g (0.0711 mol) of lutetium chloride was added. The resulting solution was stirred at room temperature for 3 hours and then evaporated under vacuum to obtain a brown liquid, which was then purified by distillation at 170° C. and 55 mTorr, thus obtaining an orange liquid. The yield was 22.4 g (64.5%). The ¹H NMR result is as shown in FIG. 28. The following characteristic peaks were obtained.

¹H NMR (C₆D₆, 25° C.): 0.83 (t, 3H), 0.98 (t, 6H), 1.19 (q, 6H), 1.93 (q, 2H), 2.44 (q, 4H), 3.00 (q, 4H), 6.0 (broad, 8H)

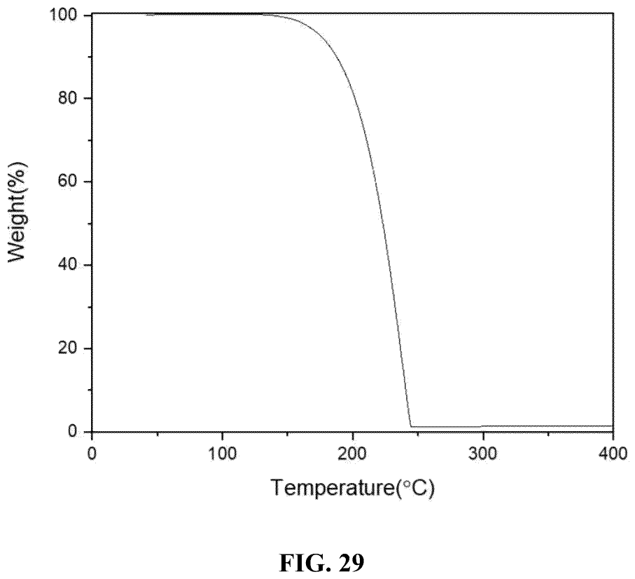

Additionally, during TGA measurement (SDT Q600 from TA Instruments) performed at a temperature ramp rate of 10° C./min in a nitrogen atmosphere at a flow rate of 200 mL/min, the orange liquid had a T_{1/2} value of 223° C. and a residual mass of 1.4% at 350° C., indicating that there were almost no residues left. This result is illustrated in FIG. 29, which shows the TGA result indicating the percentage of weight loss as a function of temperature.

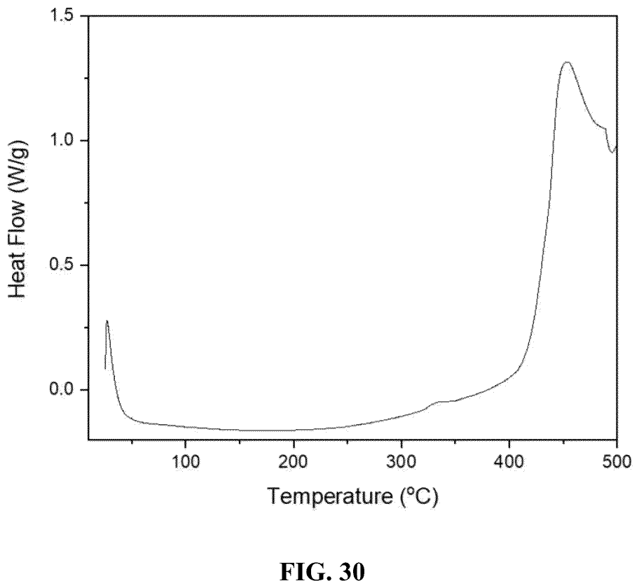

Furthermore, the orange liquid sample was placed in a sealed container for DSC and maintained at 40° C. for 10 minutes. Subsequently, a decomposition peak was observed at 455° C. during DSC measurement (Discovery 25 from TA Instruments) performed at a temperature ramp rate of 10° C./min. This result is illustrated in FIG. 30, which shows the DSC result indicating the heat energy change as a function of temperature.

The orange liquid was further analyzed using a viscometer (DV2T viscometer from Brookfield Ametek) in a nitrogen atmosphere for viscosity measurement, and was confirmed to exhibit a low viscosity of 38 cP at 25° C.

### Example 15: Synthesis of tris(diethyl-n-propylamidinato)yttrium [Y(Et₂nPr-AMD)₃]

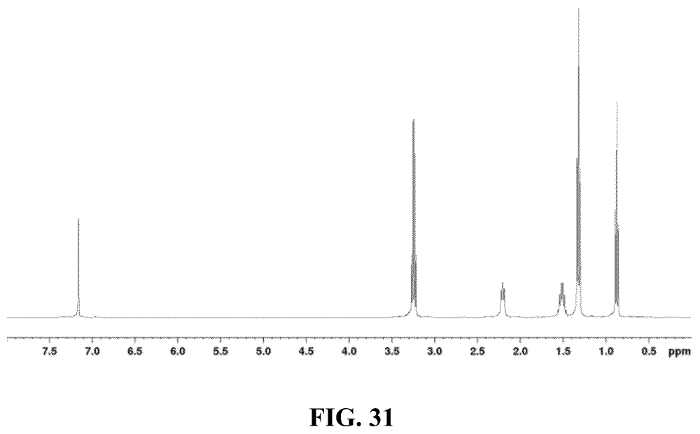

To 300 mL of THF, 109.28 g (0.768 mol) of diethyl-n-propylamidinate was added, followed by cooling to −78° C., and 307.3 mL (0.768 mol) of an n-BuLi hexane solution (2.5 M) was then slowly added dropwise, thus preparing Li—(Et₂nPr-AMD). The resulting solution was stirred at −78° C. for 30 minutes, heated to room temperature, and then stirred for an additional 2 hours at room temperature. The prepared Li—(Et₂nPr-AMD) solution was slowly added dropwise to a flask containing YCl₃ at −78° C. and then stirred at room temperature for 6 hours. The resulting mixture was evaporated under vacuum, followed by dissolution in 250 mL of pentane, filtration, and evaporation of the solvent and volatiles under vacuum to produce a red liquid, which was then treated at 184° C. and 31 mTorr, thus obtaining a pale yellow liquid. The yield was 107.5 g (81.9%). The NMR result is as shown in FIG. 31.

¹H NMR (C₆D₆, 25° C.): 0.87 (t, 9H), 1.32 (t, 18H), 1.52 (q, 6H), 2.21 (q, 6H), 3.24 (q, 12H)

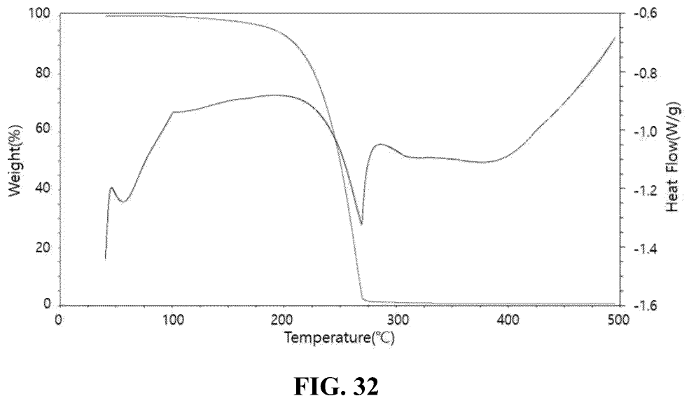

During TGA measurement performed at a temperature ramp rate of 10° C./min in a nitrogen atmosphere at a flow rate of 200 mL/min, the pale yellow liquid had a residual mass of 0.75%, indicating that there were almost no residues left. This result was confirmed by the TGA result (FIG. 32), which shows the percentage of weight loss as a function of temperature.

### Example 16: Synthesis of tris-(diethyl-n-propylamidinato)terbium [Tb(Et₂nPr-AMD)₃]

To a 500 mL Schlenk flask, 10.0 g (0.0377 mol) of TbCl₃ and 50 mL of THF were added, followed by stirring at room temperature for 4 hours. Subsequently, 50 mL of THF and 16.1 g (0.114 mol) of diethyl-n-propylamidinate were added to a 250 mL Schlenk flask, followed by cooling to −78° C. Then, 47.5 mL (0.119 mol) of an n-BuLi-hexane solution (2.5 M) was slowly added dropwise, followed by stirring at room temperature for 2 hours, thus preparing Li—(Et₂nPr-AMD). The prepared Li—(Et₂nPr-AMD) solution was added dropwise to the flask containing 10 g (0.0377 mol) of TbCl₃ at 0° C., followed by stirring at room temperature for 6 hours. The resulting mixture was filtered, followed by evaporation of the solvent and volatiles from the filtrate under vacuum. The unevaporated residues were distilled at 220° C. and 138 mTorr, thus obtaining a yellow liquid. The yield was 11.0 g (50.1%).

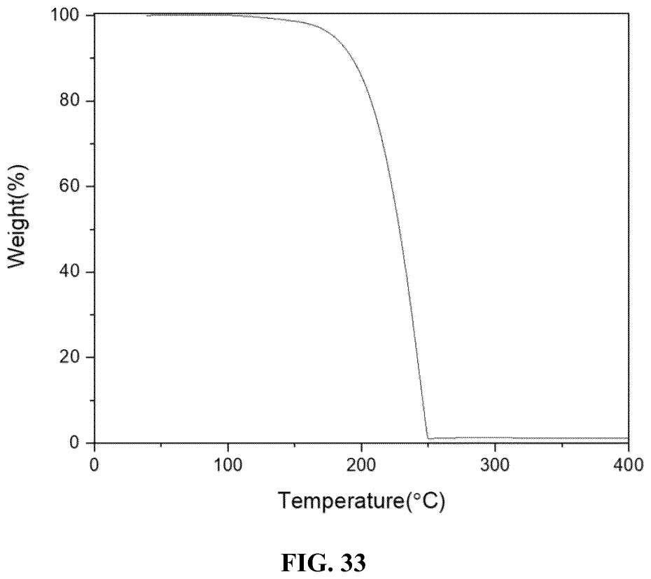

During TGA measurement (SDT Q600 from TA Instruments) performed at a temperature ramp rate of 10° C./min in a nitrogen atmosphere at a flow rate of 200 mL/min, the yellow liquid had a residual mass of 1.18%, indicating that there were almost no residues left. This result was confirmed by the TGA result in FIG. 33, which shows the percentage of weight loss as a function of temperature.

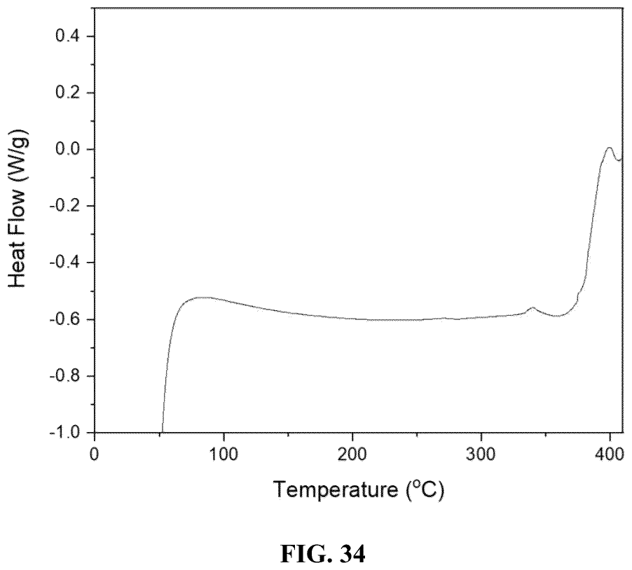

Additionally, the yellow liquid sample was placed in a sealed container for DSC and maintained at 40° C. for 10 minutes. Subsequently, a decomposition peak was observed at 417° C. during DSC measurement (Discovery 25 from TA Instruments) performed at a temperature ramp rate of 10° C./min. This result was confirmed by the DSC result in FIG. 34, which shows the heat energy change as a function of temperature.

### Example 17: Synthesis of tris-(diethyl-n-propylamidinato)dysprosium [Dy(Et₂nPr-AMD)₃]

To a 500 mL Schlenkflask, 10.0 g (0.0372 mol) of DyCl₃ and 50 mL of THF were added, followed by stirring at room temperature for 4 hours. Subsequently, 50 mL of THF and 15.9 g (0.112 mol) of diethyl-n-propylamidinate were added to a 250 mL Schlenk flask, followed by cooling to −78° C. Then, 46.9 mL (0.117 mol) of an n-BuLi-hexane solution (2.5 M) was slowly added dropwise, followed by stirring at room temperature for 2 hours, thus preparing Li—(Et₂nPr-AMD). The prepared Li—(Et₂nPr-AMD) solution was added dropwise to the flask containing 10 g (0.0372 mol) of DyCl₃ at 0° C., followed by stirring at room temperature for 6 hours. The resulting mixture was filtered, followed by evaporation of the solvent and volatiles from the filtrate under vacuum. The unevaporated residues were distilled at 220° C. and 90 mTorr, thus obtaining a yellow liquid. The yield was 13.8 g (69%).

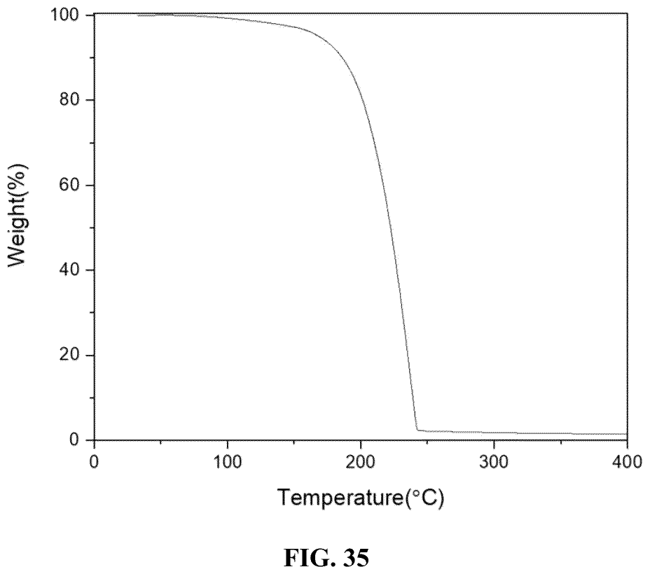

During TGA measurement (SDT Q600 from TA Instruments) performed at a temperature ramp rate of 10° C./min in a nitrogen atmosphere at a flow rate of 200 mL/min, the yellow liquid had a residual mass of 1.59%, indicating that there were almost no residues left. This result was confirmed by the TGA result in FIG. 35, which shows the percentage of weight loss as a function of temperature.

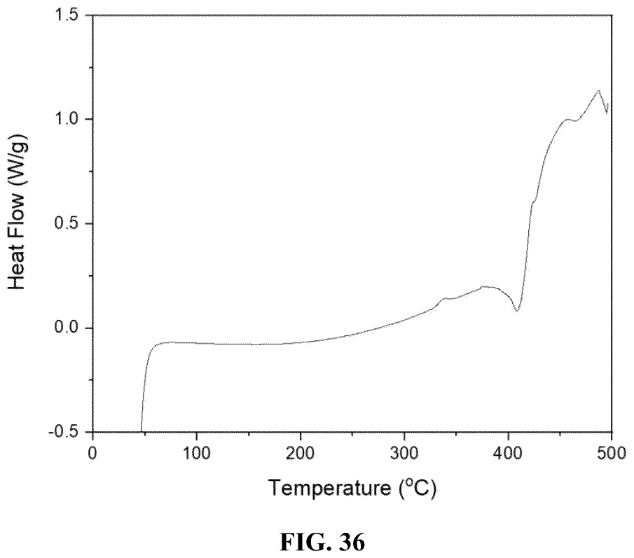

Additionally, the yellow liquid sample was placed in a sealed container for DSC and maintained at 40° C. for 10 minutes. Subsequently, a decomposition peak was observed at 487° C. during DSC measurement (Discovery 25 from TA Instruments) performed at a temperature ramp rate of 10° C./min. This result was confirmed by the DSC result in FIG. 36, which shows the heat energy change as a function of temperature.

### Example 18: Synthesis of tris-(diethyl-n-propylamidinato)erbium [Er(Et₂nPr-AMD)₃]

To a 500 mL Schlenk flask, 10.0 g (0.0365 mol) of ErCl₃ and 50 mL of THF were added, followed by stirring at room temperature for 4 hours. Subsequently, 50 mL of THF and 15.6 g (0.110 mol) of diethyl-n-propylamidinate were added to a 250 mL Schlenk flask, followed by cooling to −78° C. Then, 46.0 mL (0.115 mol) of an n-BuLi-hexane solution (2.5 M) was slowly added dropwise, followed by stirring at room temperature for 2 hours, thus preparing Li—(Et₂nPr-AMD). The prepared Li—(Et₂nPr-AMD) solution was added dropwise to the flask containing 10 g (0.0365 mol) of ErCl₃ at 0° C., followed by stirring at room temperature for 6 hours. The resulting mixture was filtered, followed by evaporation of the solvent and volatiles from the filtrate under vacuum. The unevaporated residues were then distilled at 220° C. and 80 mTorr, thus obtaining an orange liquid. The yield was 14 g (72%).

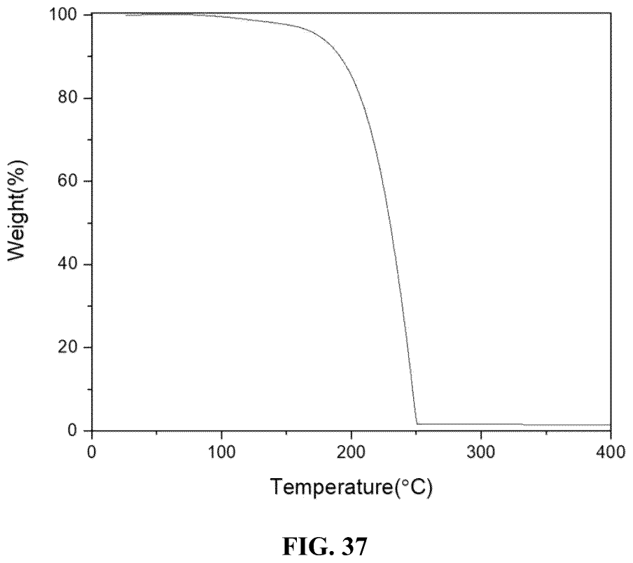

During TGA measurement (SDT Q600 from TA Instruments) performed at a temperature ramp rate of 10° C./min in a nitrogen atmosphere at a flow rate of 200 mL/min, the orange liquid had a residual mass of 1.52%, indicating that there were almost no residues left. This result was confirmed by the TGA result in FIG. 37, which shows the percentage of weight loss as a function of temperature.

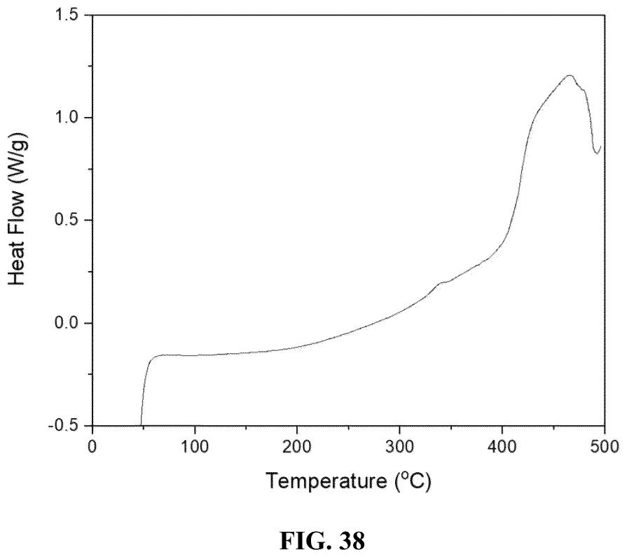

Additionally, the orange liquid sample was placed in a sealed container for DSC and maintained at 40° C. for 10 minutes. Subsequently, a decomposition peak was observed at 466° C. during DSC measurement (Discovery 25 from TA Instruments) performed at a temperature ramp rate of 10° C./min. This result was confirmed by the DSC result in FIG. 38, which shows the heat energy change as a function of temperature.

To further examine the viscosity of the orange liquid, this sample was placed in a measurement container of a rotational viscometer (LVD2T from Brookfield) for viscosity measurement at 25° C. using a low-viscosity spindle. The viscosity was found to be 77.1 cPs.

### Example 19: Synthesis of tris-(diethyl-n-propylamidinato)ytterbium [Yb(Et₂nPr-AMD)₃]

To a 500 mL Schlenk flask, 10.0 g (0.0358 mol) of YbCl₃ and 50 mL of THF were added, followed by stirring at room temperature for 4 hours. Subsequently, 50 mL of THF and 15.3 g (0.107 mol) of diethyl-n-propylamidinate were added to a 250 mL Schlenk flask, followed by cooling to −78° C. Then, 45.1 mL (0.113 mol) of an n-BuLi-hexane solution (2.5 M) was slowly added dropwise, followed by stirring at room temperature for 2 hours, thus preparing Li—(Et₂nPr-AMD). The prepared Li—(Et₂nPr-AMD) solution was added dropwise to the flask containing 10 g (0.0358 mol) of YbCl₃ at 0° C., followed by stirring at room temperature for 6 hours. The resulting mixture was filtered, followed by evaporation of the solvent and volatiles from the filtrate under vacuum. The unevaporated residues were then distilled at 220° C. and 68 mTorr, thus obtaining a yellow liquid. The yield was 14 g (70%).

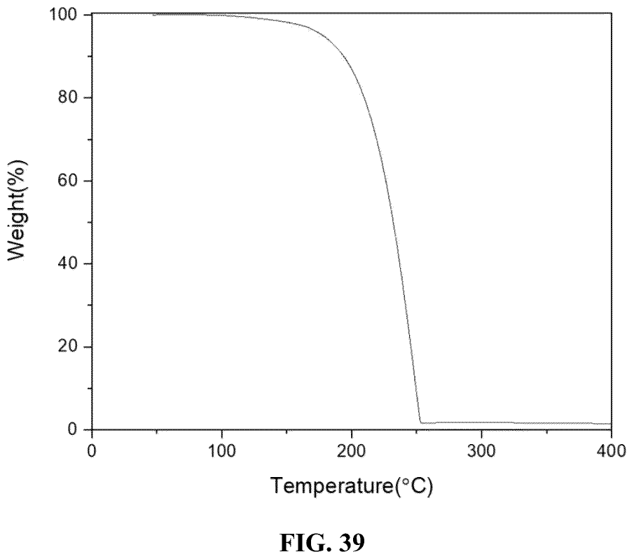

During TGA measurement (SDT Q600 from TA Instruments) performed at a temperature ramp rate of 10° C./min in a nitrogen atmosphere at a flow rate of 200 mL/min, the yellow liquid had a residual mass of 1.68%, indicating that there were almost no residues left. This result was confirmed by the TGA result in FIG. 39, which shows the percentage of weight loss as a function of temperature.

To further examine the viscosity of the yellow liquid, this sample was placed in a measurement container of a rotational viscometer (LVD2T from Brookfield) for viscosity measurement at 25° C. using a low-viscosity spindle. The viscosity was found to be 66.2 cPs.

### Example 20: Synthesis of tris-(diethyl-n-propylamidinato)lutetium [Lu(Et₂nPr-AMD)₃]

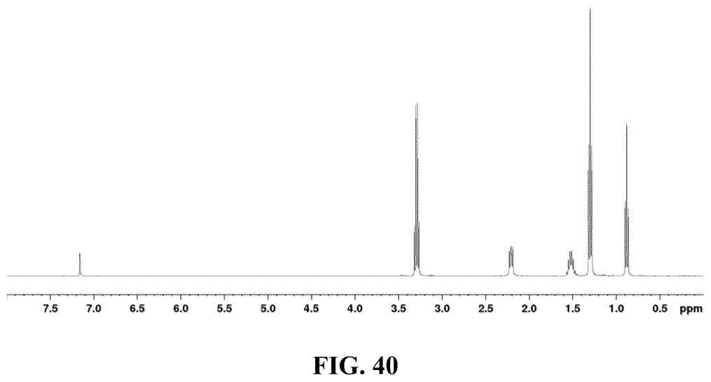

To a 500 mL Schlenk flask, 10.0 g (0.0355 mol) of LuCl₃ and 50 mL of THF were added, followed by stirring at room temperature for 4 hours. Subsequently, 50 mL of THF and 15.2 g (0.107 mol) of diethyl-n-propylamidinate were added to a 250 mL Schlenk flask, followed by cooling to −78° C. Then, 44.8 mL (0.112 mol) of an n-BuLi-hexane solution (2.5 M) was slowly added dropwise, followed by stirring at room temperature for 2 hours, thus preparing Li—(Et₂nPr-AMD). The prepared Li—(Et₂nPr-AMD) solution was added dropwise to the flask containing 10 g (0.0355 mol) of LuCl₃ at 0° C., followed by stirring at room temperature for 6 hours. The resulting mixture was filtered, followed by evaporation of the solvent and volatiles from the filtrate under vacuum. The unevaporated residues were then distilled at 220° C. and 54 mTorr, thus obtaining an orange liquid. The yield was 16.2 g (75.8%). The ¹H NMR result is as shown in FIG. 40.

¹H NMR (C₆D₆, 25° C.): 0.88 (t, 9H), 1.30 (t, 18H), 1.51 (q, 6H), 2.21 (q, 6H), 3.28 (q, 12H).

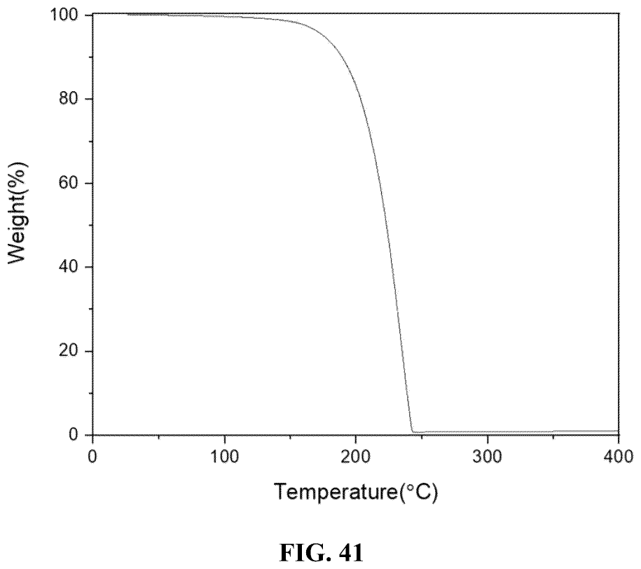

During TGA measurement (SDT Q600 from TA Instruments) performed at a temperature ramp rate of 10° C./min in a nitrogen atmosphere at a flow rate of 200 mL/min, the orange liquid had a residual mass of 0.95%, indicating that there were almost no residues left. This result was confirmed by the TGA result in FIG. 41, which shows the percentage of weight loss as a function of temperature.

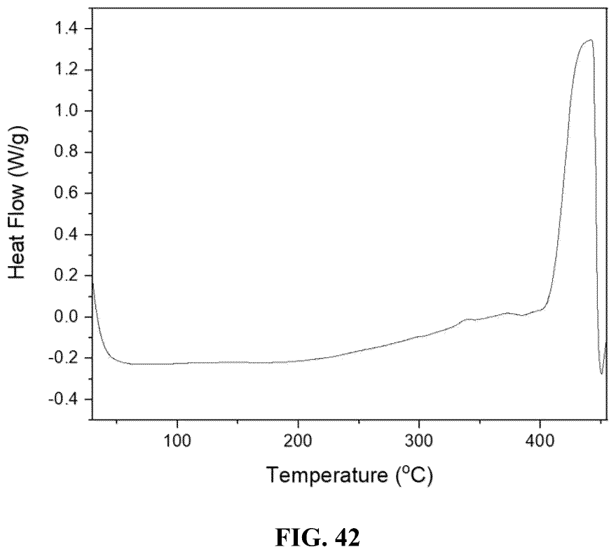

Additionally, the orange liquid sample was placed in a sealed container for DSC and maintained at 40° C. for 10 minutes. Subsequently, a decomposition peak was observed at 442° C. during DSC measurement (Discovery 25 from TA Instruments) performed at a temperature ramp rate of 10° C./min. This result was confirmed by the DSC result in FIG. 42, which shows the heat energy change as a function of temperature.

### Example 21: Preparation of (CH₂CH₂CH₂N—C(CH₂CH₃)=N—CH₂CH₂CH₃)₂Hf(DMA)₂

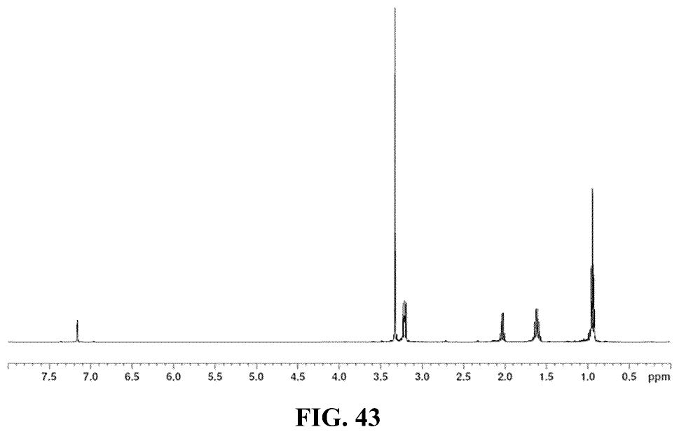

In a nitrogen atmosphere, 2.8 g (0.0080 mol) of tetrakis(dimethylamino)hafnium (Hf(NMe₂)₄) and 50 mL of n-hexane were added to a 250 mL Schlenk flask. Then, 2.5 g (0.0160 mol) of (E)-N,N-dipropylpropionimidamide was slowly added dropwise at room temperature, followed by stirring at room temperature for 14 hours. After the completion of the reaction, the obtained reaction solution was subjected to reduced pressure, thereby removing the solvent and volatile byproducts. The remaining liquid was purified at 126.6° C. (at 63.4 mTorr), thus obtaining 1 g (yield: 21.7%) of Compound 1, a pale yellow viscous liquid. The ¹H NMR result is as shown in FIG. 43.

¹H NMR (C₆D₆, 25° C.): 3.326 (s, 12H), 3.23 (t, 8H), 2.00 (q, 4H), 1.64 (m, 8H), 0.96 (t, 12H), 0.93 (t, 6H).

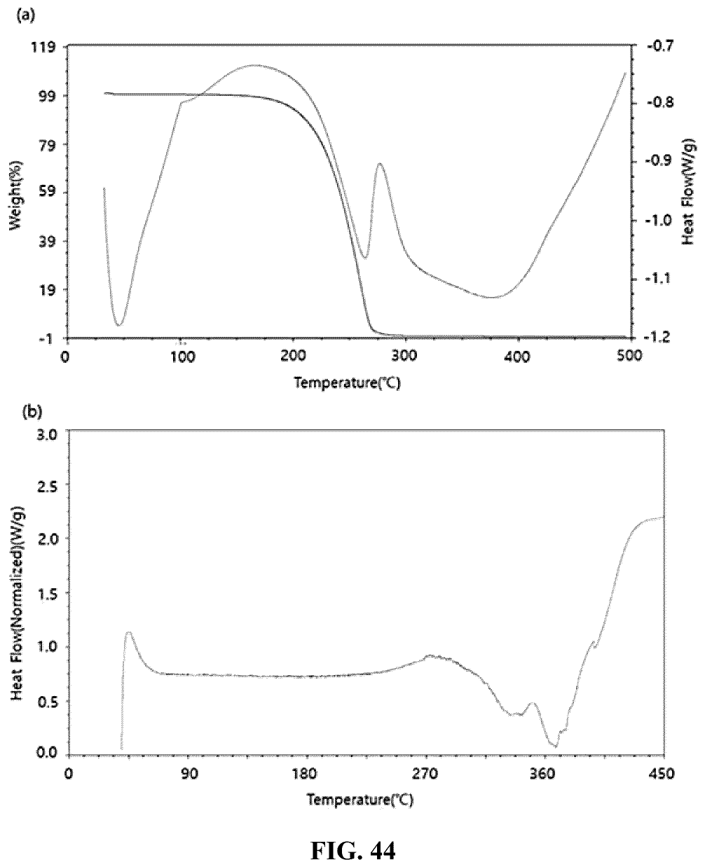

During TGA measurement (SDT Q600 from TA Instruments) performed at a temperature ramp rate of 10° C./min in a nitrogen atmosphere at a flow rate of 200 mL/min, the pale yellow liquid had a residual mass of 0.95%, indicating that there were almost no residues left. This result was confirmed by the TGA result in (a) of FIG. 44, which shows the percentage of weight loss as a function of temperature.

Additionally, the pale yellow liquid sample was placed in a sealed container for DSC and maintained at 40° C. for 10 minutes. Subsequently, a decomposition peak was observed at 351° C. during DSC measurement (Discovery 25 from TA Instruments) performed at a temperature ramp rate of 10° C./min ((b) of FIG. 44).

### Example 22: Preparation of (CH₂CH₂N—C(CH₂CH₂CH₃)=N—CH₂CH₃)₂Hf(DMA)₂

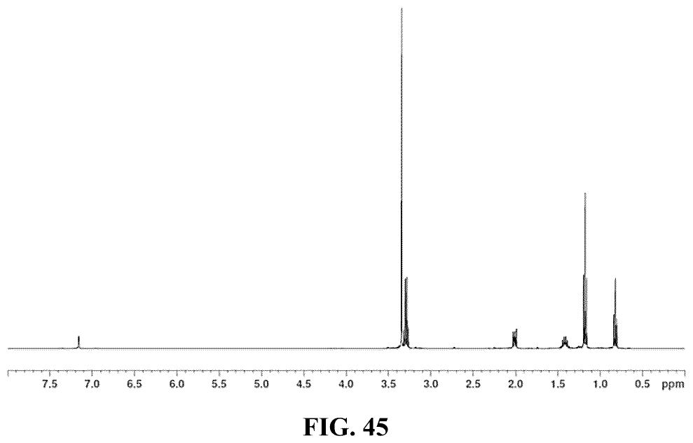

In a nitrogen atmosphere, 3.1 g (0.0088 mol) of tetrakis(dimethylamino)hafnium (Hf(NMe₂)₄) and 50 mL of n-hexane were added to a 250 mL Schlenk flask. Then, 2.5 g (0.0176 mol) of (E)-N,N-diethylpropyl butyrimidamide was slowly added dropwise at room temperature, followed by stirring at room temperature for 14 hours. After the completion of the reaction, the obtained reaction solution was subjected to reduced pressure, thereby removing the solvent and volatile byproducts. The remaining liquid was purified at 117.2° C. (at 54.3 mTorr), thus obtaining 2 g (yield: 41.7%) of Compound 3, a pale yellow liquid. The ¹H NMR result is as shown in FIG. 45.

¹H NMR (C₆D₆, 25° C.): 3.345 (s, 12H), 3.31 (q, 8H), 2.02 (m, 4H), 1.44 (m, 4H), 1.19 (t, 12H), 0.83 (t, 6H).

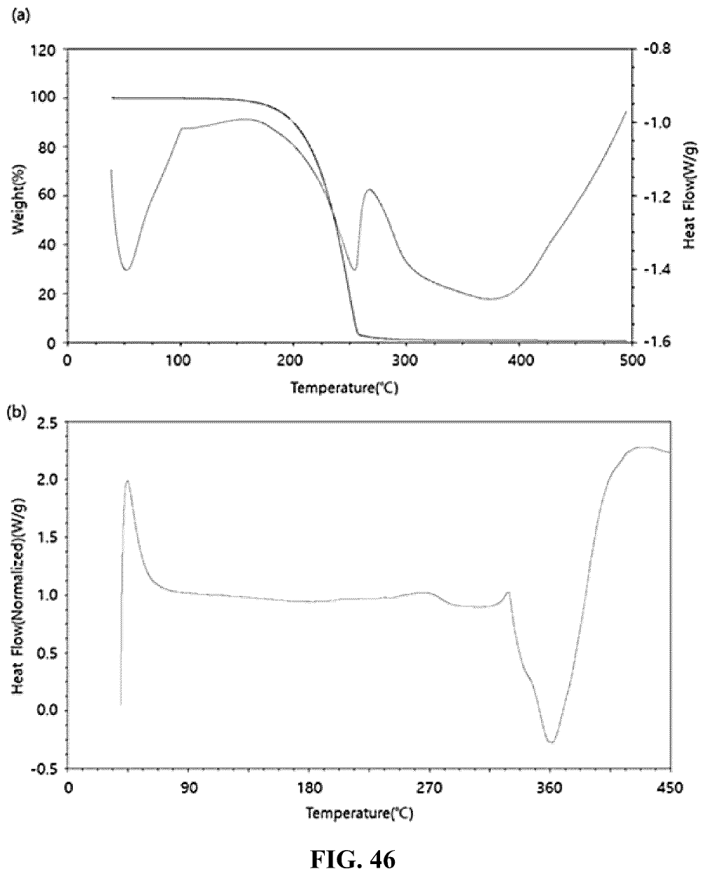

During TGA measurement (SDT Q600 from TA Instruments) performed at a temperature ramp rate of 10° C./min in a nitrogen atmosphere at a flow rate of 200 mL/min, the pale yellow liquid had a residual mass of 0.53%, indicating that there were almost no residues left. This result was confirmed by the TGA result in (a) of FIG. 46, which shows the percentage of weight loss as a function of temperature.

Additionally, the pale yellow sample was placed in a sealed container for DSC and maintained at 40° C. for 10 minutes. Subsequently, a decomposition peak was observed at 330° C. during DSC measurement (Discovery 25 from TA Instruments) performed at a temperature ramp rate of 10° C./min ((b) of FIG. 46).

### Example 23: Preparation of (CH₂CH₂N—C(CH₂CH₂CH₃)=N—CH₂CH₃)₂-f(EMA)₂

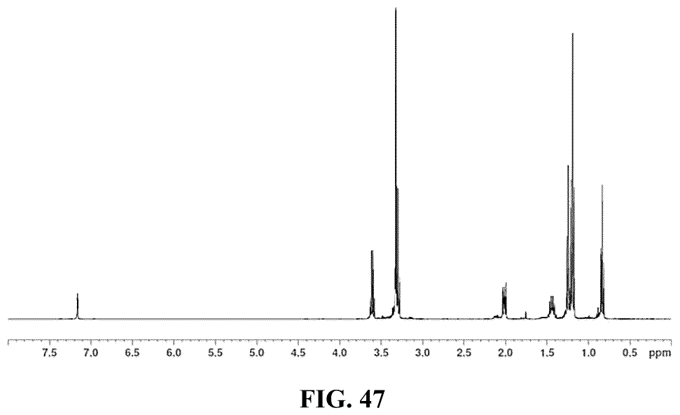

In a nitrogen atmosphere, 3.6 g (0.0080 mol) of tetrakis(ethylmethylamino)hafnium (Hf(NEtMe)₄) and 50 mL of n-hexane were added to a 250 mL Schlenk flask. Then, 2.5 g (0.0176 mol) of (E)-N,N-diethylpropyl butyrimidamide was slowly added dropwise at room temperature, followed by stirring at room temperature for 14 hours. After the completion of the reaction, the obtained reaction solution was subjected to reduced pressure, thereby removing the solvent and volatile byproducts. The remaining liquid was purified at 123.4° C. (at 25.3 mTorr), thus obtaining 2 g (yield: 39.2%) of Compound 4, a pale yellow liquid. The ¹H NMR result is as shown in FIG. 47.

¹H NMR (C₆D₆, 25° C.): 3.63 (q, 4H), 3.33 (q, 8H), 3.32 (s, 6H), 2.20 (m, 4H), 1.46 (m, 4H), 1.25 (q, 6H), 1.20 (t, 12H), 0.08 (t, 61).

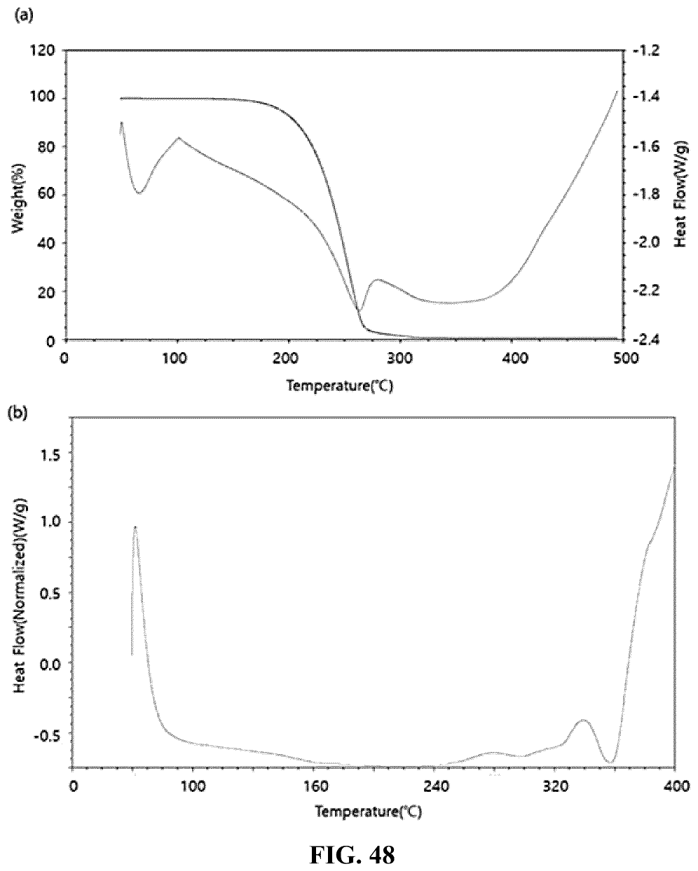

During TGA measurement (SDT Q600 from TA Instruments) performed at a temperature ramp rate of 10° C./min in a nitrogen atmosphere at a flow rate of 200 mL/min, the pale yellow liquid had a residual mass of 0.95%, indicating that there were almost no residues left. This result was confirmed by the TGA result in (a) of FIG. 48, which shows the percentage of weight loss as a function of temperature.

Additionally, the pale yellow liquid sample was placed in a sealed container for DSC and maintained at 40° C. for 10 minutes. Subsequently, a decomposition peak was observed at 339° C. during DSC measurement (Discovery 25 from TA Instruments) performed at a temperature ramp rate of 10° C./min ((b) of FIG. 48).

### Example 24: Preparation of (CH₂CH₂N—C(CH₂CH₂CH₃)=N—CH₂CH₃)₂Zr(DMA)₂

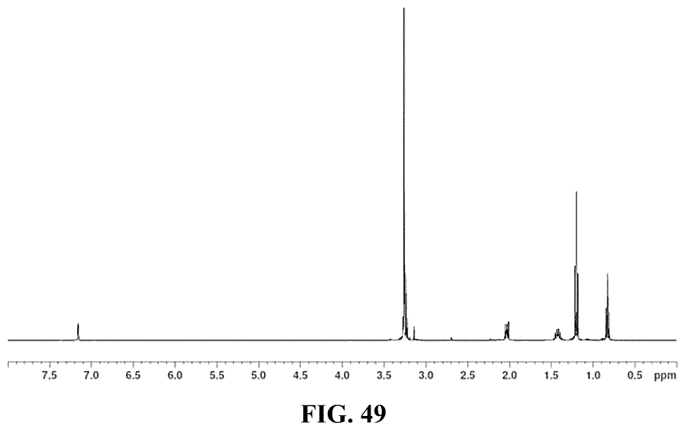

In a nitrogen atmosphere, 4 g (0.0150 mol) of tetrakis(dimethylamino)zirconium (Zr(NMe₂)₄) and 50 mL of n-hexane were added to a 250 mL Schlenk flask. Then, 4.3 g (0.0300 mol) of (E)-N,N′-diethylpropyl butyrimidamide was slowly added dropwise at room temperature, followed by stirring at room temperature for 14 hours. After the completion of the reaction, the obtained reaction solution was subjected to reduced pressure, thereby removing the solvent and volatile byproducts. The remaining liquid was purified at 116.2° C. (at 67.8 mTorr), thus obtaining 2.9 g (yield: 42.0%) of Compound 5, a pale yellow liquid. The ¹H NMR result is as shown in FIG. 49.

¹H NMR (C₆D₆, 25° C.): 3.27 (q, 8H), 3.261 (s, 12H), 2.05 (m, 4H), 1.45 (m, 4H), 1.21 (t, 12H), 0.84 (t, 6H)

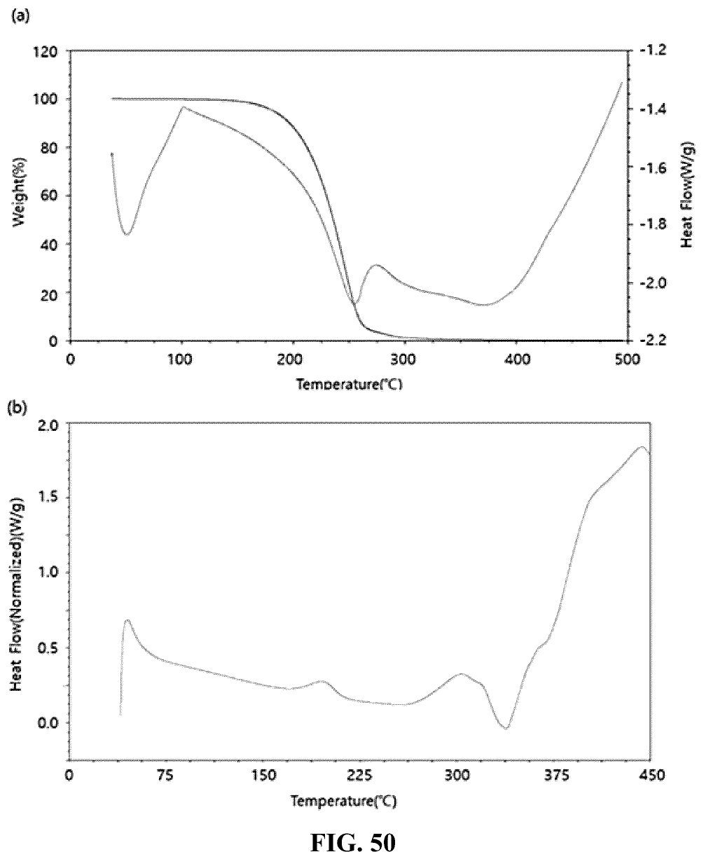

During TGA measurement (SDT Q600 from TA Instruments) performed at a temperature ramp rate of 10° C./min in a nitrogen atmosphere at a flow rate of 200 mL/min, the pale yellow liquid had a residual mass of 0.95%, indicating that there were almost no residues left. This result was confirmed by the TGA result in (a) of FIG. 50, which shows the percentage of weight loss as a function of temperature.

Additionally, the pale yellow liquid sample was placed in a sealed container for DSC and maintained at 40° C. for 10 minutes. Subsequently, a decomposition peak was observed at 304° C. during DSC measurement (Discovery 25 from TA Instruments) performed at a temperature ramp rate of 10° C./min ((b) of FIG. 50).

### Example 25: Preparation of (2-ethyl-N,N-diethylamidinato)bis(dimethylamino)borane [(Et₂Et-AMD)B(DMA)₂]

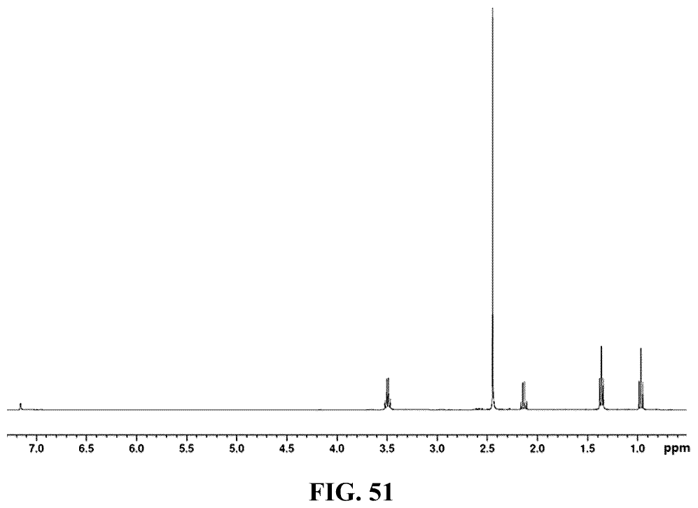

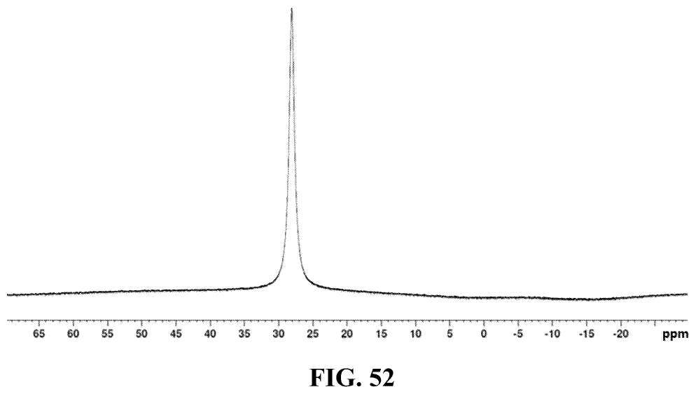

In Reactor 1, 5.9 g (0.04 mol) of tris(dimethylamino)borane (TDMAB) was diluted with 30 mL of hexane, followed by cooling to a cryogenic temperature (approximately −20° C.). Then, 2 mL (0.02 mol) of tribromoboron (BBr₃) was added, followed by stirring at room temperature for approximately 6 hours. In Reactor 2, 7.98 g (0.06 mol) of 2-ethyl-N,N-diethylamidinate was diluted with 30 mL of hexane, followed by cooling to a cryogenic temperature (approximately −20° C.). Then, 24.9 mL (0.06 mol) of 2.5 M n-butyl lithium (nBuLi) was slowly added, followed by stirring at room temperature for approximately 6 hours. After re-cooling Reactor 1 to a cryogenic temperature (approximately −20° C.), the solution in Reactor 2 was slowly transferred and added, followed by stirring at room temperature for approximately 12 hours. The resulting product was then filtered, and the solvent was removed from the obtained filtrate under reduced pressure, thus obtaining a colorless liquid. The resulting liquid was purified under reduced pressure, thus obtaining 8.5 g (yield: 61%). The ¹H NMR result is as shown in FIG. 51, and the ¹¹B NMR result is as shown in FIG. 52.

¹H NMR (C₆D₆, 25° C.): 0.96 (t, 3H), 1.36 (t, 6H), 2.13 (q, 2H), 2.44 (s, 12H), 3.49 (q, 4H).

¹¹B-NMR: 28.09 ppm.

### Example 26: Preparation of (2-methyl-N,N-diisopropylamidinato)bis(dimethylamino)borane [(iPr₂Me-AMD)B(DMA)₂]

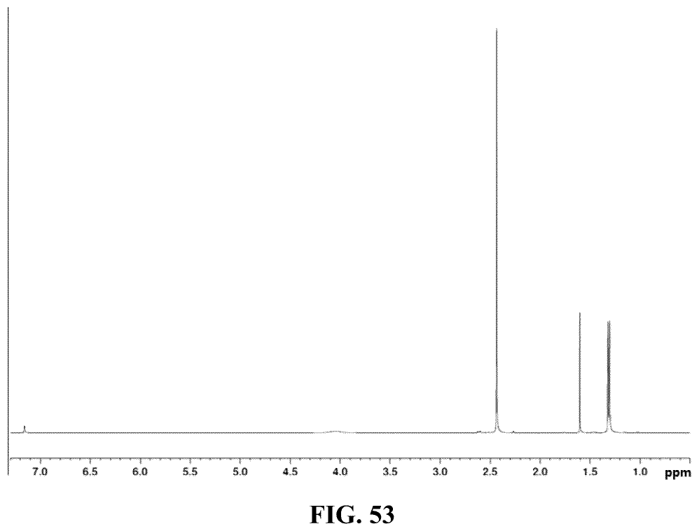

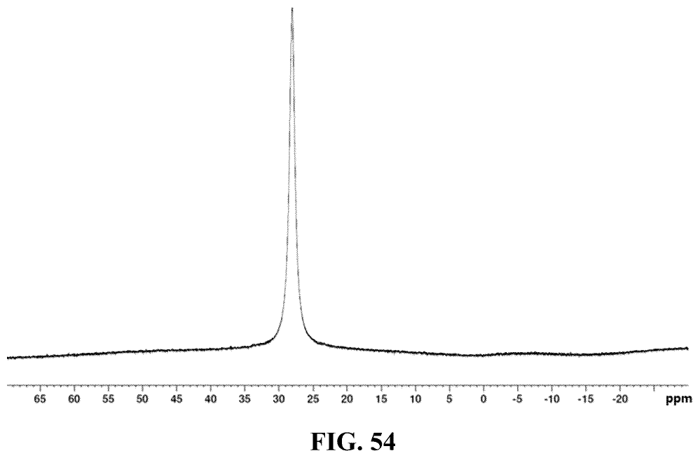

In Reactor 1, 5.9 g (0.04 mol) of TDMAB was diluted with 20 mL of hexane, followed by cooling to a cryogenic temperature (approximately −20° C.). Then, 2 mL (0.02 mol) of BBr₃ was added, followed by stirring at room temperature for approximately 6 hours. In Reactor 2, 0.86 g (0.06 mol) of N,N-diisopropylcarbodiimide was diluted with 20 mL of hexane, followed by cooling to a cryogenic temperature (approximately −20° C.). Then, 42.8 mL (0.06 mol) of 1.6 M methyl lithium (MeLi) was slowly added, followed by stirring at room temperature for approximately 6 hours. After re-cooling Reactor 1 to a cryogenic temperature (approximately −20° C.), the solution in Reactor 2 was slowly transferred and added, followed by stirring at room temperature for approximately 12 hours. The resulting product was then filtered, and the solvent was removed from the obtained filtrate under reduced pressure, thus obtaining a colorless liquid. The resulting liquid was purified under reduced pressure [at 82.5° C. and 1 Torr], thus obtaining 7.5 g (yield: 50%) of a colorless liquid. The ¹H NMR result is as shown in FIG. 53, and the ¹¹B NMR result is as shown in FIG. 54.

¹H NMR (C₆D₆, 25° C.): 1.31 (d, 12H), 1.60 (s, 3H), 2.43 (s, 12H), 4.05 (m, 2H).

¹¹B-NMR: 28.07 ppm.

### Example 27: Preparation of (2-ethyl-N,N-diethylamidinato)(N,N-dimethylethylenediamino)borane [(Et₂Et-AMD)B(DMA-EDA)]

First, 79 mL (0.56 mol) of triethylamine was diluted with 200 mL of hexane, followed by cooling to a low temperature (approximately 0° C.). Then, a solution of 26.9 mL (0.28 mol) of BBr₃ diluted with 40 mL of hexane was added, followed by stirring at room temperature for approximately 2 hours. A solution of 25 g (0.28 mol) of N,N-dimethylethylenediamine diluted with 100 mL of hexane was added and heated, followed by stirring for approximately 3 hours. The resulting product was then filtered, and the solvent was removed from the obtained filtrate under reduced pressure, thus obtaining a colorless liquid. The resulting liquid was purified under reduced pressure [at 51.5° C. and 40 torr], thus obtaining 23 g (yield: 69%) of a colorless liquid intermediate product.

¹H NMR (C₆D₆, 25° C.): 2.49 (s, 6H) 2.76 (s, 4H).

¹¹B-NMR: 26.32 ppm.

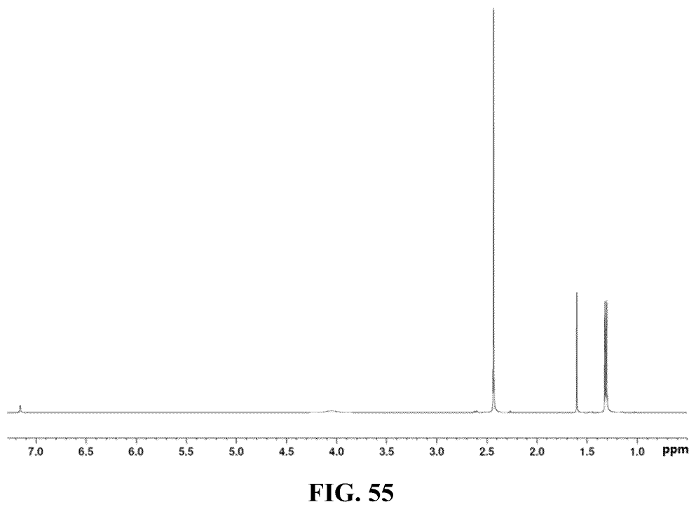

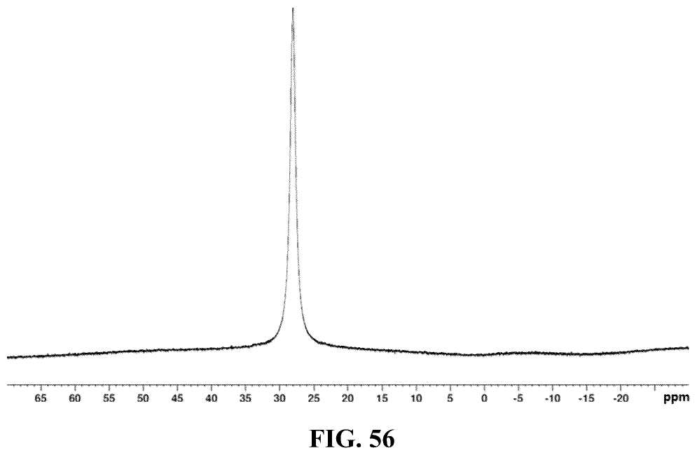

In Reactor 2, 3.62 g (0.02 mol) of 2-ethyl-N,N-diethylamidinate was diluted with 30 mL of hexane, followed by cooling to a cryogenic temperature (approximately −20° C.). Then, 11.3 mL (0.02 mol) of 2.5 M nBuLi was slowly added, followed by stirring at room temperature for approximately 6 hours. In Reactor 1, 5 g (0.02 mol) of 2-bromo-1,3-dimethyl-1,3,2-diazaborolane was diluted with 10 ml of hexane, followed by cooling to a cryogenic temperature (approximately −20° C.). Then, the solution in Reactor 2 was slowly transferred and added, followed by stirring at room temperature for approximately 12 hours. The resulting product was then filtered, and the solvent was removed from the obtained filtrate under reduced pressure, thus obtaining a colorless liquid. The resulting liquid was purified under reduced pressure [at 63.3° C. and 1 Torr], thus obtaining 3.6 g (yield: 60%) of a colorless liquid. The ¹H NMR result is as shown in FIG. 55, and the ¹¹B NMR result is as shown in FIG. 56.

¹H NMR (C₆D₆, 25° C.): 1.01 (t, 3H), 1.35 (t, 6H), 2.19 (q, 2H), 2.42 (s, 6H), 2.88 (s, 4H), 3.52 (q, 4H).

¹¹B-NMR: 28.31 ppm

### Example 28: Preparation of (N-ethyl-2-isobutyl-N-propylamidinato)bis(dimethylamino)borane [(EtPrisoBu-AMD)B(DMA)₂]

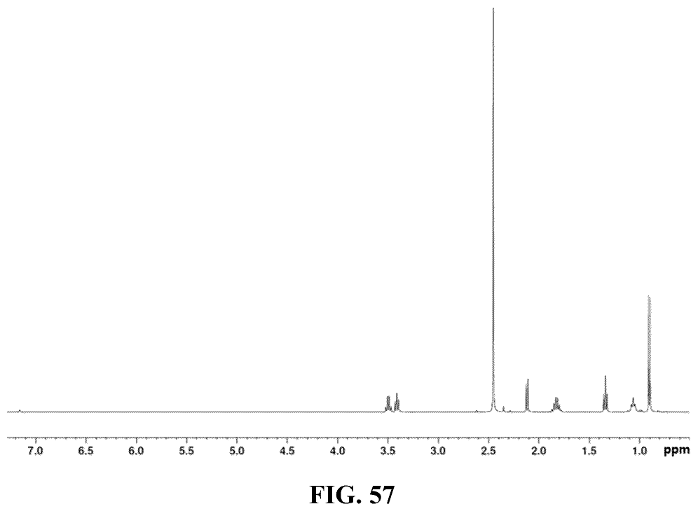

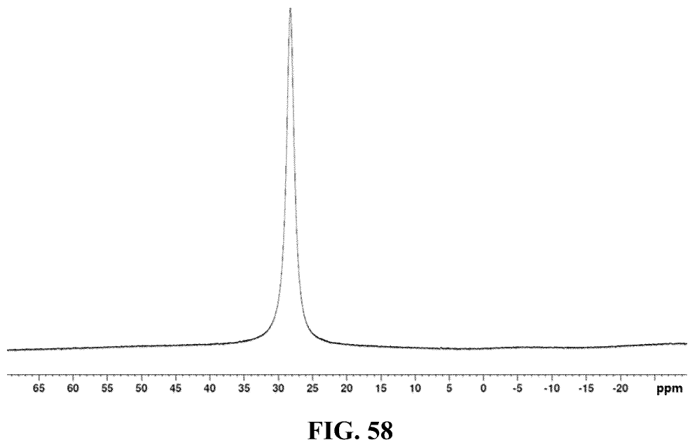

In Reactor 1, 5.9 g (0.04 mol) of TDMAB was diluted with 15 mL of hexane, followed by cooling to a cryogenic temperature (approximately −20° C.). Then, 2 mL (0.02 mol) of BBr₃ was added, followed by stirring at room temperature for approximately 6 hours. In Reactor 2, 10.6 g (0.06 mol) of N-ethyl-2-isobutyl-N-propylamidinate was diluted with 30 mL of hexane, followed by cooling to a cryogenic temperature (approximately −20° C.). Then, 24.9 mL (0.06 mol) of 2.5 M nBuLi was slowly added, followed by stirring at room temperature for approximately 6 hours. After re-cooling Reactor 1 to a cryogenic temperature (approximately −20° C.), the solution in Reactor 2 was slowly transferred and added, followed by stirring at room temperature for approximately 12 hours. The resulting product was then filtered, and the solvent was removed from the obtained filtrate under reduced pressure, thus obtaining a colorless liquid. The resulting liquid was purified under reduced pressure [at 83.7° C. and 0.1 Torr], thus obtaining 12.4 g (yield: 74%) of a colorless liquid. The ¹H NMR result is as shown in FIG. 57, and the ¹¹B NMR result is as shown in FIG. 58.

¹H NMR (C₆D₆, 25° C.): 0.90 (d, 6H), 1.06 (t, 3H), 1.33 (t, 3H), 1.82 (m, 3H), 2.11 (d, 2H), 2.45 (s, 12H), 3.41 (t, 2H), 3.49 (q, 2H).

¹¹B-NMR: 28.25 ppm.

Through these experimental results, it was confirmed that, in the structure employing the novel amidinate ligand according to the present disclosure, a liquid precursor exhibiting sufficient vapor pressure and thermal stability for use as a semiconductor precursor could be obtained, and, in particular, that the properties of the precursor, such as viscosity, were significantly improved.

The present disclosure has been described hereinabove with reference to preferred embodiments, but is not limited to these embodiments. Various modifications and changes may be made by those skilled in the art to which the present disclosure belongs without departing from the idea of the present disclosure. Such modifications and changes should be construed as falling within the scope of the present disclosure and the appended claims.

1. A precursor for forming a thin film, the precursor comprising a compound represented by Chemical Formula 1 below,
wherein the precursor is a liquid at room temperature, 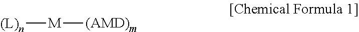
wherein in Chemical Formula 1,
AMD refers to an amidinate ligand,
M is a central metal atom and is any one of the following: a Group 2 to 6 element, a Group 13 element, a Group 15 element, a transition metal, and a rare-earth element,
L is a ligand that is the same as or different from AMD and, when being different from AMD, is any one of the following: a substituted or unsubstituted cyclopentadienyl group, amine, alcohol, alkyl, aryl, amino amine, alkoxy amine, amino alcohol, alkoxy alcohol, imido, diamine, diol, formidinate, guanidinate, β-diketonate, ketoiminate, amide, or halide,
n is an integer in a range of 0 to 5, and
m is an integer in a range of 1 to 6.

2. The precursor of claim 1, wherein AMD is a ligand represented by Chemical Formula 2 below, 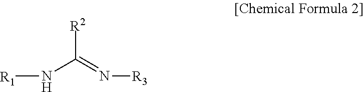
wherein in Chemical Formula 2,
R₁ and R₃ are each independently a straight-chain, branched-chain, or cyclic alkyl or alkenyl group having 1 to 5 carbon atoms, and
R₂ is a hydrogen atom, or a straight-chain, branched-chain, or cyclic alkyl or alkenyl group having 1 to 5 carbon atoms.

3. The precursor of claim 2, wherein in Chemical Formula 2, R₂ is a straight-chain, branched-chain, or cyclic alkyl or alkenyl group having 2 to 5 carbon atoms.

4. The precursor of claim 2, wherein in Chemical Formula 2, R₁ and R₃ are each independently a straight-chain, branched-chain, or cyclic alkyl or alkenyl group having 2 to 5 carbon atoms, and R₂ is a straight-chain, branched-chain, or cyclic alkyl or alkenyl group having 2 to 5 carbon atoms.

5. The precursor of claim 2, wherein in Chemical Formula 2, R₁ and R₃ are methyl groups.

6. The precursor of claim 2, wherein in Chemical Formula 2, R₂ is an isopropyl group.

7. The precursor of claim 2, wherein in Chemical Formula 2, R₁ and R₃ are each independently a straight-chain alkyl or alkenyl group having 1 to 5 carbon atoms.

8. The precursor of claim 2, wherein in Chemical Formula 2, R₁ and R₃ are each independently a straight-chain alkyl or alkenyl group having 1 to 5 carbon atoms, and R₂ is a straight-chain alkyl or alkenyl group having 1 to 5 carbon atoms.

9. The precursor of claim 2, wherein in Chemical Formula 2, R₁ and R₃ are both the same, and are straight-chain, branched-chain, or cyclic alkyl or alkenyl groups having 1 to 5 carbon atoms.

10. The precursor of claim 2, wherein in Chemical Formula 2, R₁ to R₃ are all the same, and are straight-chain, branched-chain, or cyclic alkyl or alkenyl groups having 1 to 4 carbon atoms.

11. The precursor of claim 2, wherein the amidinate ligand comprises one or more selected from the following chemical structures.

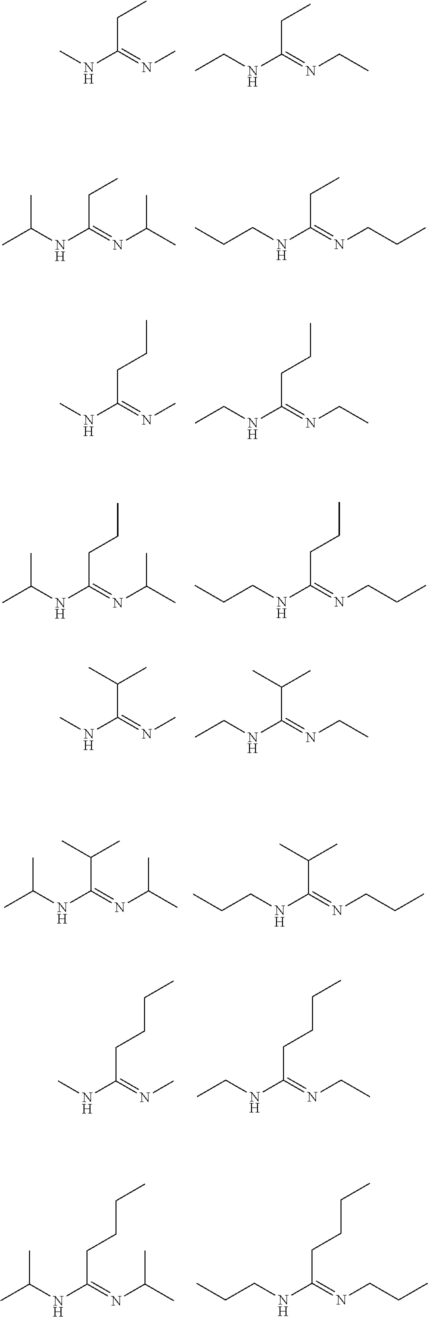

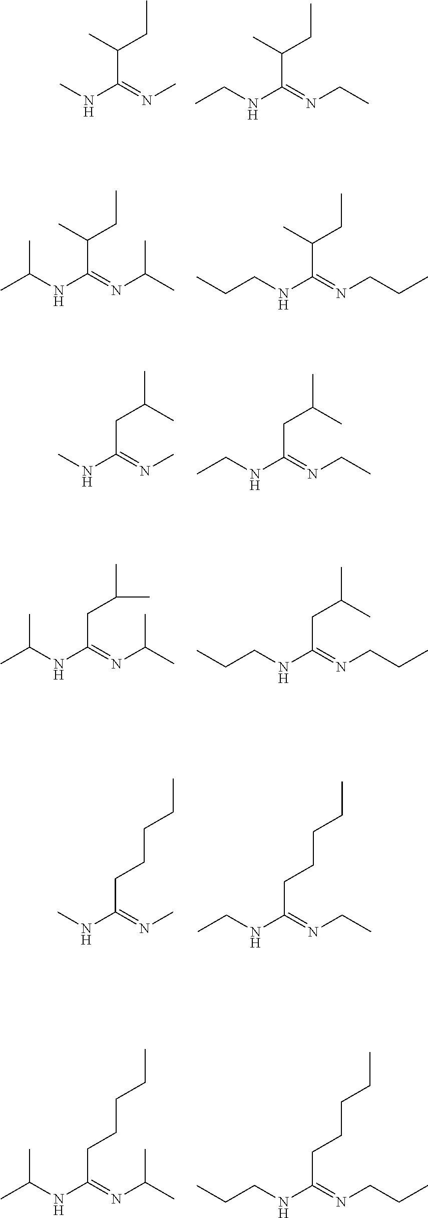

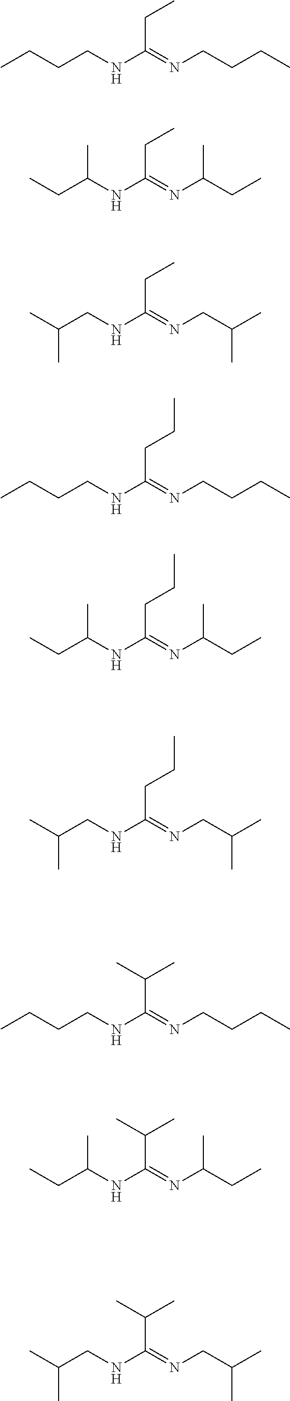

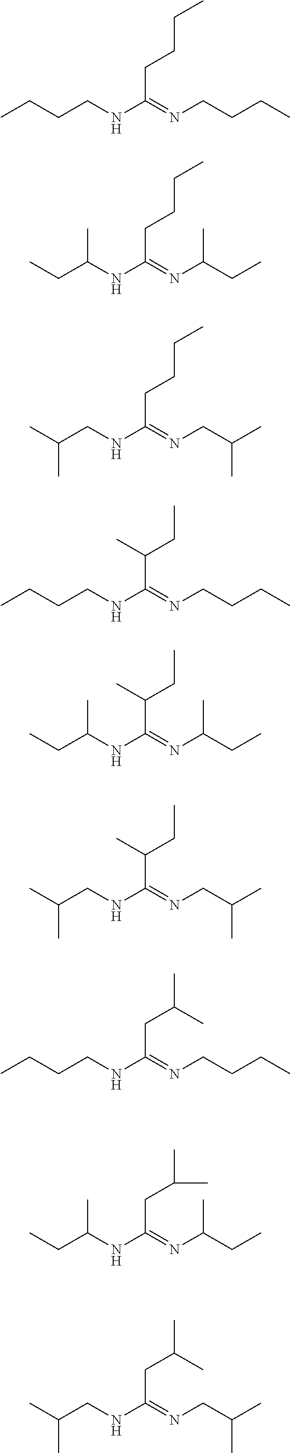

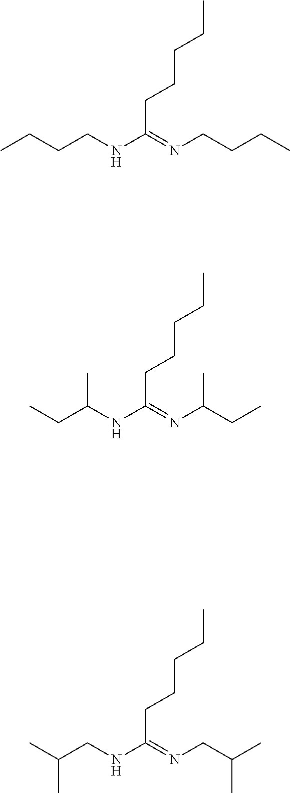

12. The precursor of claim 1, wherein the precursor has a viscosity of 60 cP or less (at a temperature of 25° C.).

13. The precursor of claim 1, wherein the compound represented by Chemical Formula 1 comprises one or more selected from the following chemical structures.

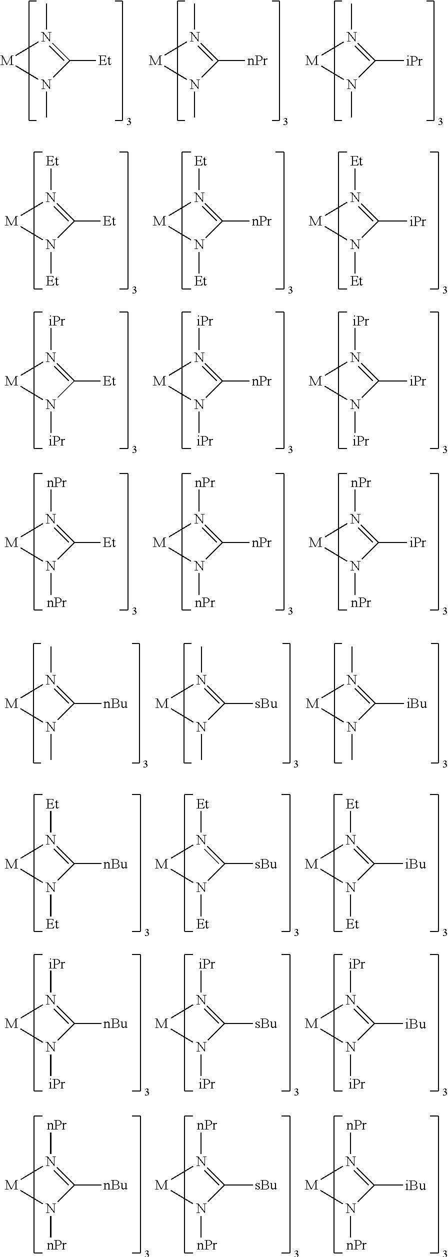

14. The precursor of claim 1, wherein the compound represented by Chemical Formula 1 comprises one or more selected from the following chemical structures.

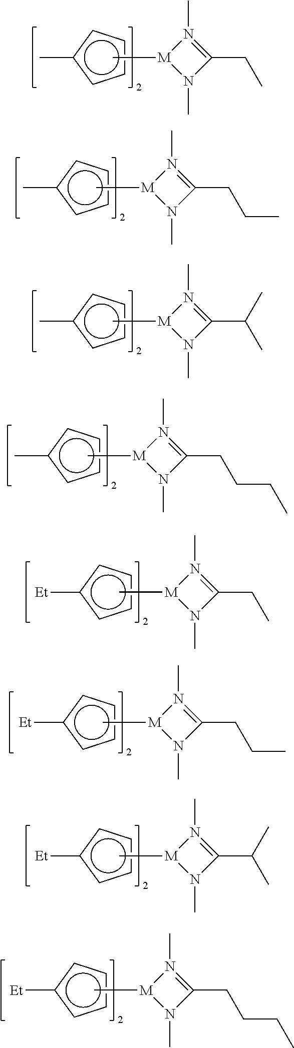

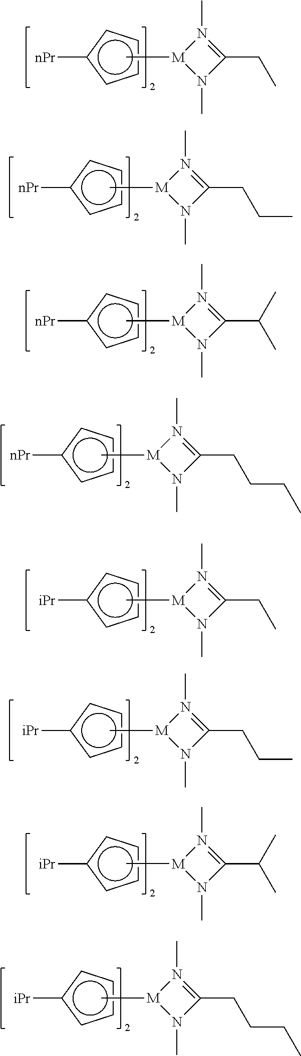

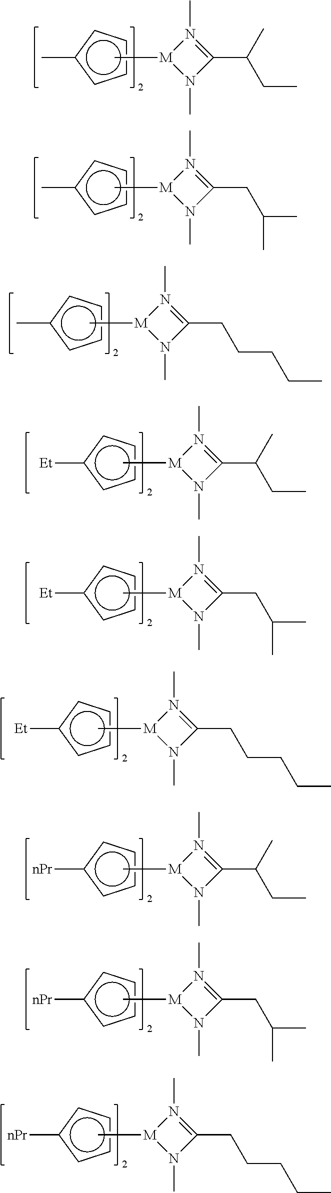

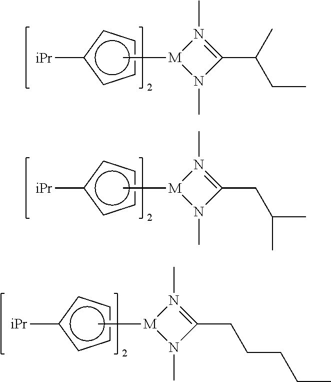

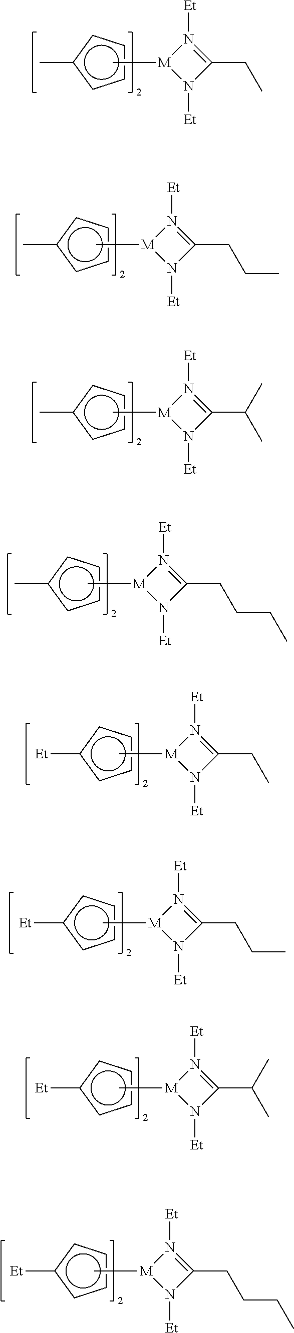

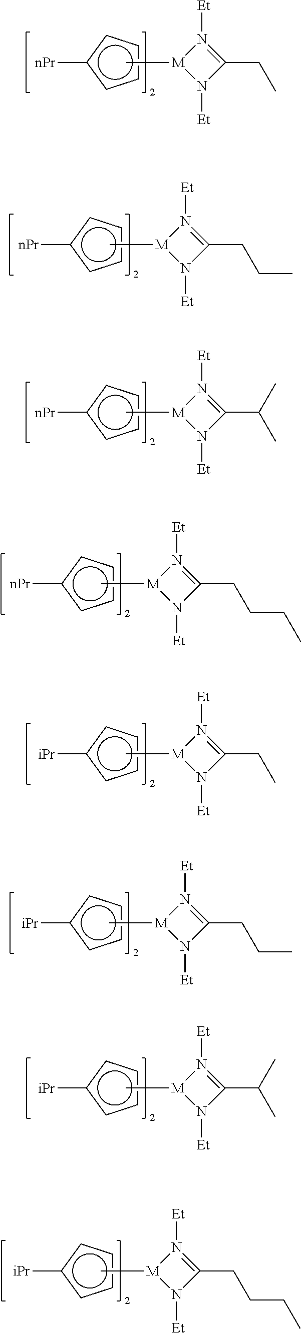

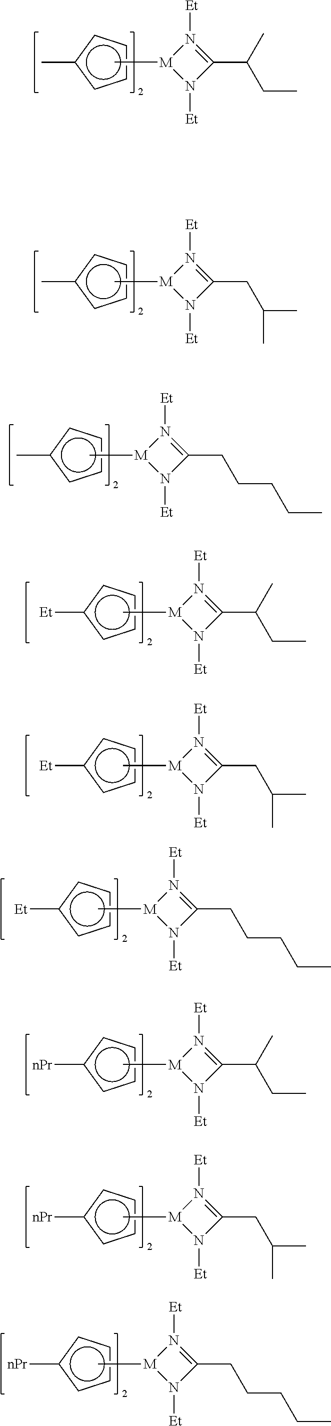

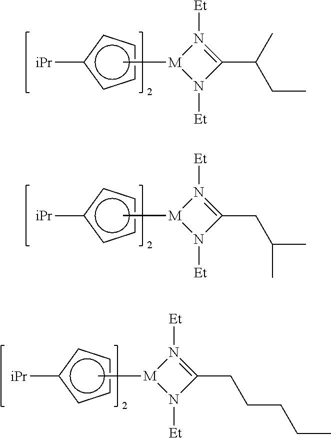

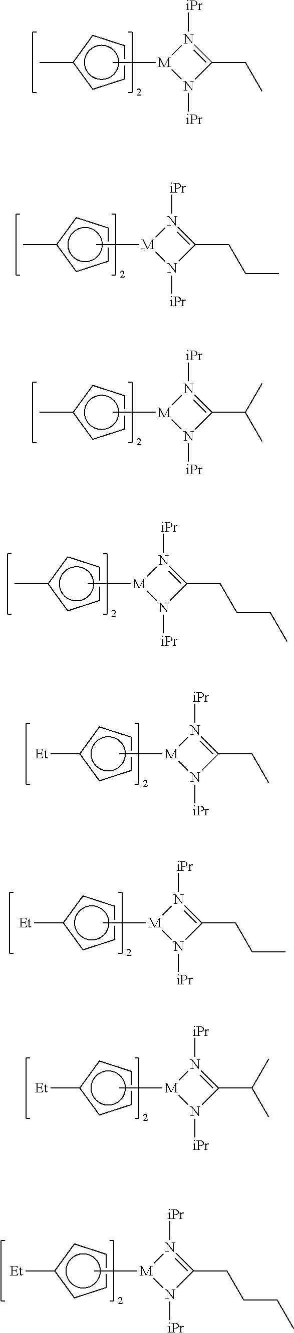

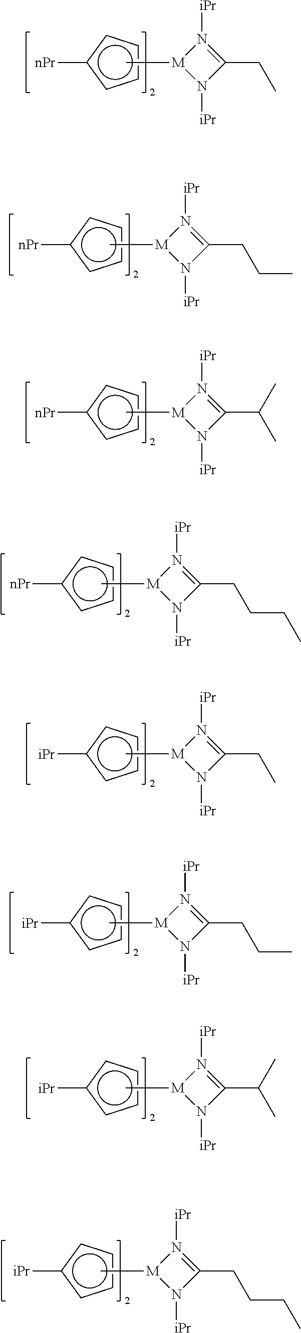

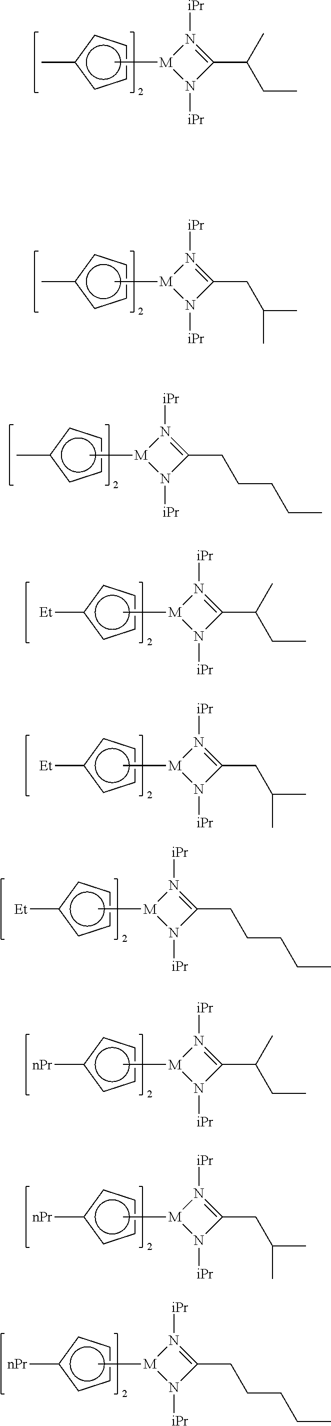

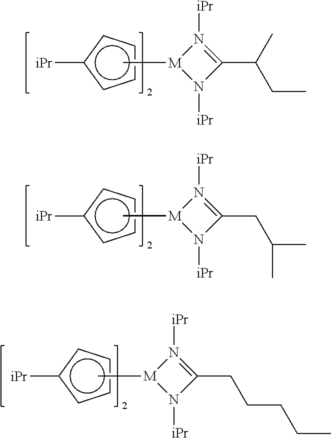

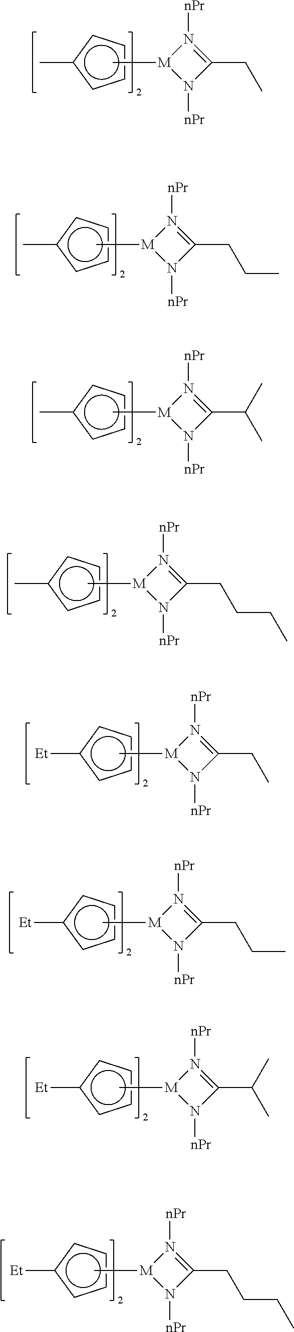

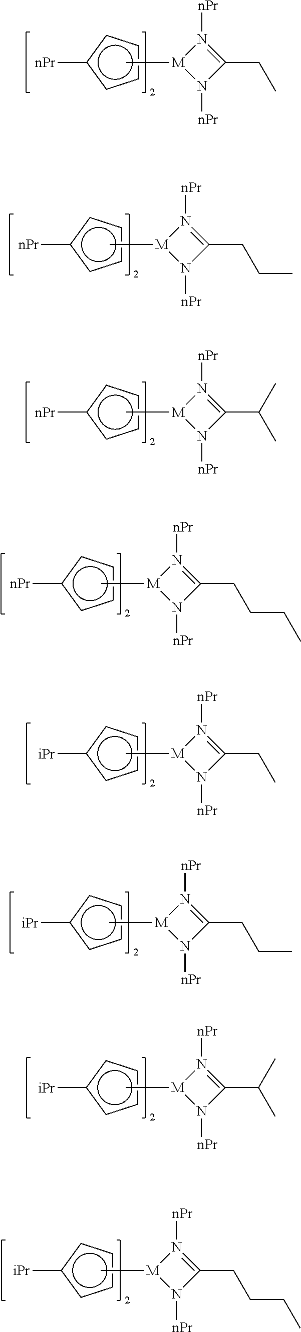

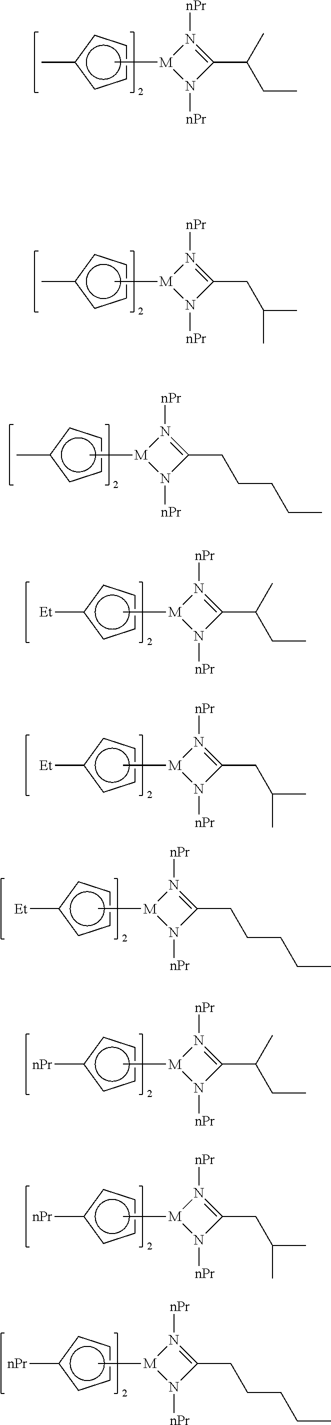

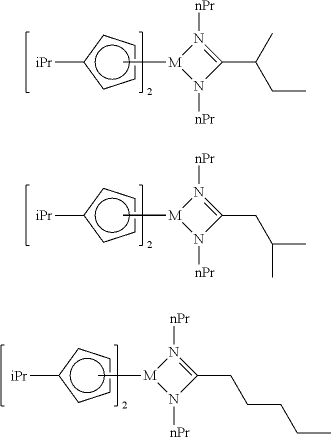

15. The precursor of claim 1, wherein the compound represented by Chemical Formula 1 comprises one or more selected from the following chemical structures. 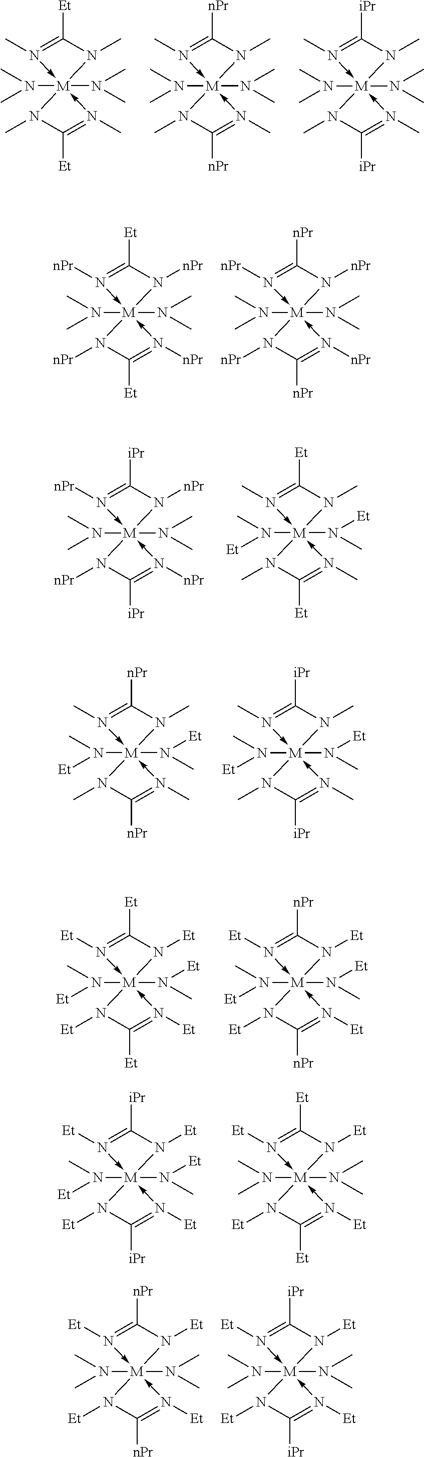 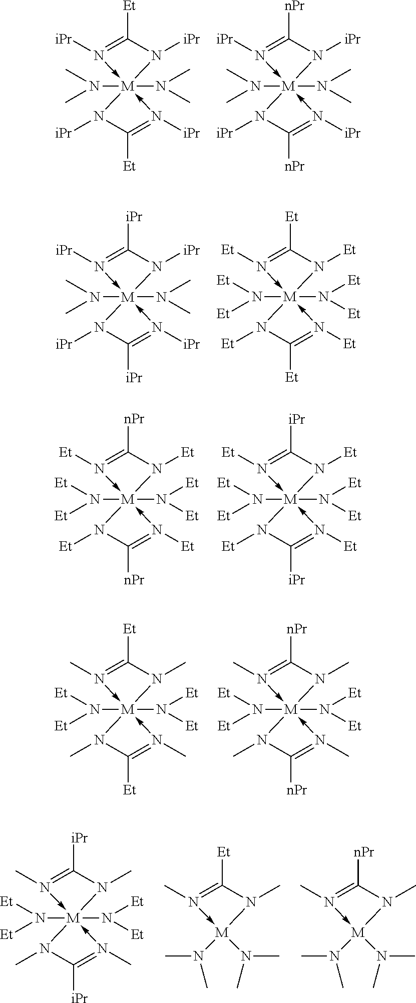 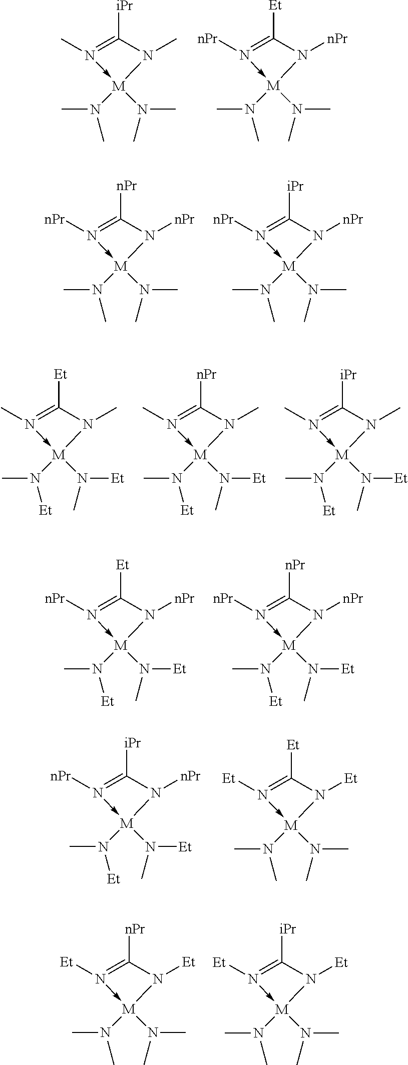 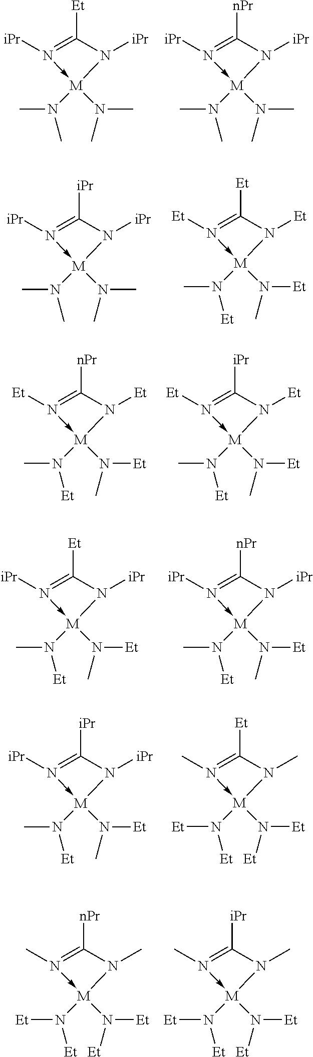 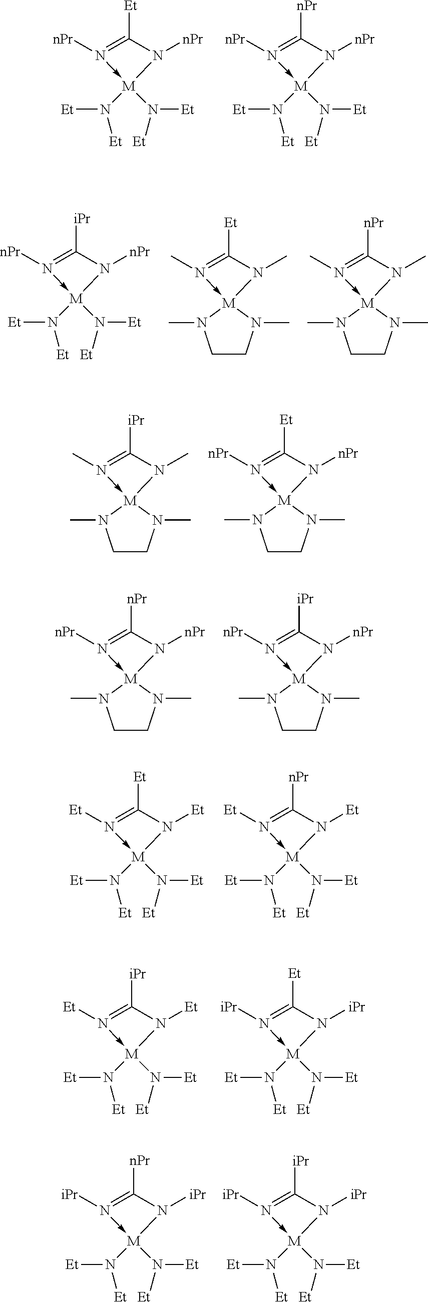 [chemical structure]
